SEQUENCE LISTING (46 sequences)

SEQ ID NO: 1 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 2 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 3 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 4 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 5 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 6 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 7 - protein, 171 aa, Nephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 8 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 9 - protein, 420 aa, Nephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 10 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 11 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 12 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 13 - protein, 32 aa, Euprosthenops australis
GGQGGQGQGRYGQGAGSSAAAAAAAAAAAAAA

SEQ ID NO: 14 - protein, 42 aa, Tetragnatha kauaiensis
GGLGGGQGAGQGGQQGAGQGGYGSGLGGAGQGASAAAAAAAA

SEQ ID NO: 15 - protein, 42 aa, Argiope aurantia
GGYGPGAGQQGPGSQGPGSGGQQGPGGLGPYGPSAAAAAAAA

SEQ ID NO: 16 - protein, 46 aa, Deinopis spinosa
GPGGYGGPGQQGPGQGQYGPGTGQQGQGPSGQQGPAGAAAAAAAAA

SEQ ID NO: 17 - protein, 42 aa, Nephila clavata
GPGGYGLGQQGPGQQGPGQQGPAGYGPSGLSGPGGAAAAAAA

SEQ ID NO: 18 - protein, 174 aa, Deinopis Spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 19 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 20 - protein, 161 aa, Nephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 21 - protein, 186 aa, Nephilengys cruentata
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 22 - protein, 132 aa, Uloborus diversus
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAA

SEQ ID NO: 23 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 24 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 25 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 26 - protein, 177 aa, Nephilengys cruentata
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 27 - protein, 174 aa, Nephilengys cruentata
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 28 - protein, 22 aa, Saccharomyces cerevisiae
MFSLKALLPLALLLVSANQVAA

SEQ ID NO: 29 - protein, 23 aa, Pichia pastoris
MSFSSNVPQLFLLLVLLTNIVSG

SEQ ID NO: 30 - protein, 20 aa, Pichia pastoris
MKLSTNLILAIAAASAVVSA

SEQ ID NO: 31 - protein, 18 aa, Gallus gallus
MRSLLILVLCFLPLAALG

SEQ ID NO: 32 - protein, 89 aa, Artificial Sequence
  Description of Artificial Sequence Synthetic polypeptide
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 33 - protein, 89 aa, Artificial Sequence
  Description of Artificial Sequence Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 34 - protein, 92 aa, Artificial Sequence
  Description of Artificial Sequence Synthetic polypeptide
MFSLKALLPLALLLVSANQVAAAPVNTTTEDETAQIPAEAVIGYSDLEGDFDVAVLPFSNSTNNGLLFINTTIASIAAKEEGVSLEKREAEA

SEQ ID NO: 35 - protein, 93 aa, Artificial Sequence
  Description of Artificial Sequence Synthetic polypeptide
MSFSSNVPQLFLLLVLLTNIVSGAPVNTTTEDETAQIPAEAVIGYSDLEGDFDVAVLPFSNSTNNGLLFINTTIASIAAKEEGVSLEKREAEA

SEQ ID NO: 36 - protein, 90 aa, Artificial Sequence
  Description of Artificial Sequence Synthetic polypeptide
MKLSTNLILAIAAASAVVSAAPVNTTTEDETAQIPAEAVIGYSDLEGDFDVAVLPFSNSTNNGLLFINTTIASIAAKEEGVSLEKREAEA

SEQ ID NO: 37 - protein, 88 aa, Artificial Sequence
  Description of Artificial Sequence Synthetic polypeptide
MRSLLILVLCFLPLAALGAPVNTTTEDETAQIPAEAVIGYSDLEGDFDVAVLPFSNSTNNGLLFINTTIASIAAKEEGVSLEKREAEA

SEQ ID NO: 38 - protein, 943 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAA

SEQ ID NO: 39 - protein, 800 aa, Artificial Sequence
  Description of Artificial Sequence Synthetic polypeptide
MAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSAGGGGGG

SEQ ID NO: 40 - protein, 24 aa, Artificial Sequence
  Description of Artificial Sequence Synthetic peptide
DYKDDDDKDYKDDDDKDYKDDDDK

SEQ ID NO: 41 - protein, 5 aa, Unknown
  Description of Unknown silk protein sequence
AAAAA

SEQ ID NO: 42 - protein, 5 aa, Unknown
  Description of Unknown silk protein sequence
SGAGG

SEQ ID NO: 43 - protein, 5 aa, Unknown
  Description of Unknown silk protein sequence
GSGAG

SEQ ID NO: 44 - protein, 5 aa, Unknown
  Description of Unknown silk protein sequence
GGSGA

SEQ ID NO: 45 - protein, 8 aa, Artificial Sequence
  Description of Artificial Sequence Synthetic peptide
  MISC_FEATURE (1)..(8): This sequence may encompass 6-8 residues
HHHHHHHH

SEQ ID NO: 46 - protein, 6 aa, Artificial Sequence
  Description of Artificial Sequence Synthetic 6xHis tag
HHHHHH

(19) United States
(12) Patent Application Publication
(10) Pub. No.: US 2022/0017580 A1
(43) Pub. Date: Jan. 20, 2022

(54) ALKALINE PURIFICATION OF SPIDER SILK PROTEINS

(71) Applicant: Bolt Threads, Inc., Emeryville, CA (US)

(72) Inventors: Phillip MUI, Fremont, CA (US); Ritu Bansal MUTALIK, Albany, CA (US); Simon LI, San Francisco, CA (US); Scott CHAN, San Leandro, CA (US)

(21) Appl. No.: 17/297,787

(22) PCT Filed: Nov. 26, 2019
(86) PCT No.: PCT/US2019/063208

§ 371 Date: May 27, 2021

Related U.S. Application Data
(60) Provisional application No. 62/772,588, filed on Nov. 28, 2018.

(51) Int. Cl. C07K 14/435; C07K 1/34; C07K 1/36; C07K 1/14

(52) U.S. Cl. CPC C07K 14/43518; C07K 1/145; C07K 1/36; C07K 1/34

(57) ABSTRACT

The present disclosure relates to methods of producing and purifying synthetic block copolymer proteins, expression constructs for their secretion, recombinant microorganisms for their production, and synthetic fibers comprising these proteins that recapitulate many properties of natural silk.

### RELATED APPLICATIONS

This application claims priority to, and the benefit of, U.S. Provisional Patent Application No. 62/772,588, filed Nov. 28, 2018, the contents of which are hereby incorporated by reference in their entirety.

### SEQUENCE LISTING

The instant application contains a Sequence Listing which has been submitted via EFS-Web and is hereby incorporated by reference in its entirety. Said ASCII copy, created on Month XX, 20XX, is named XXXXXUS_sequencelisting.txt, and is X,XXX,XXX bytes in size.

### BACKGROUND OF THE INVENTION

Spider's silk polypeptides are large (>150 kDa, >1000 amino acids) polypeptides that can be broken down into three domains: an N-terminal non-repetitive domain (NTD), the repeat domain (REP), and the C-terminal non-repetitive domain (CTD). The NTD and CTD are relatively small (˜150, ˜100 amino acids respectively), well-studied, and are believed to confer to the polypeptide aqueous stability, pH sensitivity, and molecular alignment upon aggregation. NTD also has a strongly predicted secretion tag, which is often removed during heterologous expression. The repetitive region composes ˜90% of the natural polypeptide, and folds into the crystalline and amorphous regions that confer strength and flexibility to the silk fiber, respectively.

Silk polypeptides come from a variety of sources, including bees, moths, spiders, mites, and other arthropods. Some organisms make multiple silk fibers with unique sequences, structural elements, and mechanical properties. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

Currently, recombinant silk fibers are not commercially available and, with a handful of exceptions, are not produced in microorganisms outside of Escherichia coli and other gram-negative prokaryotes. Recombinant silks produced to date have largely consisted either of polymerized short silk sequence motifs or fragments of native repeat domains, sometimes in combination with NTDs and/or CTDs. This has resulted in the production of small scales of recombinant silk polypeptides (milligrams at lab scale, kilograms at bioprocessing scale) produced using intracellular expression and purification by chromatography or bulk precipitation. These methods do not lead to viable commercial scalability that can compete with the price of existing technical and textile fibers. Additional production hosts that have been utilized to make silk polypeptides include transgenic goats, transgenic silkworms, and plants. These hosts have yet to enable commercial scale production of silk, presumably due to slow engineering cycles and poor scalability.

Additionally, recombinant silk polypeptides form undesirable insoluble aggregates during production and purification. Methods to re-solubilize the peptides during purification often degrade the proteins, resulting in poor yield and fibers with low tenacity and poor hand feel. In addition, standard protein solubilization methods require the use of chaotropes such as urea, guanidine-HCl, or guanidine thiocyanate, which must be collected and disposed of properly after protein isolation. Improved methods to purify these polypeptides in a sustainable and environmentally friendly process are therefore required.

### SUMMARY OF THE INVENTION

In one aspect, provided herein are methods of isolating recombinant spider silk protein from a host cell culture, comprising obtaining a cell culture, wherein said cell culture comprises a host cell and a growth medium, wherein said host cell expresses recombinant spider silk protein, collecting a portion of said cell culture comprising said recombinant spider silk protein, incubating said portion of said cell culture in an aqueous solution under alkaline conditions, thereby solubilizing said recombinant spider silk protein in said aqueous solution, and isolating the recombinant spider silk protein from said aqueous solution, thereby producing an isolated recombinant spider silk protein sample

In some embodiments, the alkaline conditions comprise an alkaline pH from 9 to 14. In one embodiment, the alkaline pH is from 11 to 12.

In some embodiments, the isolated recombinant spider silk protein is a full-length recombinant spider silk protein. In one embodiment, the isolated recombinant spider silk protein sample comprises at least 30%, at least 35%, at least 40%, at least 45%, at least 50%, at least 55%, at least 60%, at least 65%, at least 70%, at least 75%, at least 80%, at least 85%, at least 90%, or at least 95% full-length recombinant spider silk protein as compared to total isolated recombinant spider silk protein. In one embodiment, the percentage of full-length recombinant spider silk protein is measured using a Western blot. In another embodiment, the percentage of full-length recombinant spider silk protein is measured using Size Exclusion Chromatography.

In some embodiments, the purity of the isolated recombinant spider silk protein is 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 09-95%, or 95-100%. In some embodiments, the yield of the isolated recombinant spider silk protein is at least 50-55%, 55-60%, 60-65%, 65-70%, 70-75%, 75-80%, 80-85%, 85-90%, 09-95%, or 95-100% as compared to recombinant spider silk isolated by a urea or a guanidine thiocyanate method.

In some embodiments, isolating the recombinant spider silk protein comprises precipitating the recombinant spider silk protein by altering said alkaline conditions of said aqueous solution. In one embodiment, altering said alkaline conditions comprises adjusting the alkaline pH of the portion of the cell culture to a lowered pH from 4 to 10. In one embodiment, the lowered pH is a pH of 4, 5, 6, 7, 8, 9, or 10. In on embodiment, the lowered pH is a pH from 6 to 7.

In some embodiments, adjusting the alkaline pH comprises adding an acid to the aqueous solution. In one embodiment, the acid is H₂SO₄.

In some embodiments, the portion of said cell culture comprises a supernatant, a whole cell broth, or a cell pellet. In some embodiments, collecting said portion of said cell culture comprises removing said host cell from said growth medium and reconstituting said host cell in said aqueous solution.

In some embodiments, collecting said portion of said cell culture comprises lysing said host cell. In various embodiments, lysing comprises heat treatment, shear disruption, physical homogenization, sonication, or chemical homogenization.

In some embodiments, said portion of said cell culture comprises said host cell and said growth medium from said cell culture.

In various embodiments, said aqueous solution comprises diluted growth medium.

In some embodiments, wherein incubating said portion of said cell culture under alkaline conditions is performed from 10 to 120 minutes. In some embodiments, incubating said portion of said cell culture under alkaline conditions is performed for at least 10, at least 15, at least 30, at least 45, at least 60, at least 75, at least 90, at least 105, or at least 120 minutes. In some embodiments, incubating said portion of said cell culture under alkaline conditions is performed from 15 to 30 minutes.

In various embodiments, incubating said portion of said cell culture under alkaline conditions further comprises agitating the portion of the cell culture.

In various embodiments, the method further comprises removing an un-solubilized biomass from said aqueous solution under alkaline conditions. In some embodiments, removing the un-solubilized biomass comprises filtration, centrifugation, gravitational settling, adsorption, dialysis, or phase separation. In some embodiments, the filtration is ultrafiltration, microfiltration, or diafiltration. In some embodiments, wherein removing the un-solubilized biomass is repeated at least once.

In various embodiments, the method further comprises removing impurities before isolating the recombinant spider silk protein or after isolating the recombinant spider silk protein. In some embodiments, removing the impurities comprises filtration, centrifugation, gravitational settling, adsorption, dialysis, or phase separation. In various embodiments, the filtration is ultrafiltration, microfiltration, or diafiltration. In some embodiments, the centrifugation is ultracentrifugation or diacentrifugation. In one embodiment, the adsorption is charcoal adsorption. In some embodiments, removing impurities is repeated at least once.

In various embodiments, the method further comprises concentrating the isolated recombinant spider silk protein to produce a concentrated spider silk protein. In some embodiments, concentrating comprises precipitation, filtration, ultrafiltration, centrifugation, dialysis, evaporation, or lyophilization.

In various embodiments, the method further comprises drying the isolated recombinant spider silk protein.

In various embodiments, the method further comprises generating a silk fiber from the isolated recombinant spider silk. In one embodiment, said silk fiber comprises a tenacity of at least 19 cN/tex.

In some embodiments, said recombinant spider silk protein is 18B or P0.

In some embodiments, the cell culture comprises a fungal, a bacterial or a yeast cell.

In some embodiments, the yeast cell is a Pichia pastoris cell.

In another aspect, provided herein are methods of isolating a recombinant spider silk protein, the method comprising obtaining a cell culture, wherein said cell culture comprises a host cell and a growth medium, wherein said host cell expresses a recombinant spider silk protein, collecting a portion of said cell culture comprising said recombinant spider silk protein, incubating said portion of said cell culture in an aqueous solution under alkaline conditions, thereby solubilizing said recombinant spider silk protein in said aqueous solution, adjusting the aqueous solution to a non-alkaline pH, thereby precipitating the said solubilized recombinant spider silk protein, and isolating the recombinant spider silk protein from said portion of cell culture, thereby producing an isolated recombinant spider silk protein.

In another aspect, provided herein are compositions comprising a recombinant spider silk protein produced by any one of the disclosed methods.

In some embodiments, the recombinant spider silk comprises at least 55%, at least 60%, at least 65%, at least 70%, at least 75%, at least 80%, at least 85%, at least 90%, at least 95%, or 100% full length recombinant spider silk.

In another aspect, provided herein are silk fibers comprising a recombinant spider silk protein produced by any one of the disclosed methods.

In some embodiments, the silk fiber comprises a tenacity of at least 19 cN/tex.

In another aspect, provided herein are compositions comprising a cell culture comprising a growth medium and a host cell comprising a recombinant spider silk protein in an alkaline buffer solution.

In one embodiment, the alkaline buffer solution has a pH from 9 to 14. In another embodiment, the pH is from 11 to 12.

In some embodiments, the spider silk protein is 18B or P0. In some embodiments, the cell culture comprises a fungal, a bacterial, or a yeast cell. In one embodiment, the bacterial cell is an E. coli cell. In one embodiment, the yeast cell is a Pichia pastoris cell.

### BRIEF DESCRIPTION OF THE SEVERAL VIEWS OF THE DRAWINGS

FIG. 1 shows an exemplary process flow for isolating recombinant spider silk proteins from cell supernatant.

FIG. 2 shows an exemplary process flow for isolating recombinant spider silk proteins from cell lysate.

FIG. 3 shows an exemplary process flow for isolating recombinant spider silk proteins using a chaotrope.

FIG. 4A shows the size exclusion chromatography (SEC) analysis of purified 18B spider silk protein isolated from a cell pellet using an alkaline pH buffer. The 18B monomer peak is indicated by the arrow. FIG. 4B shows a comparison of the amount and purity of 18B spider silk as purified using a urea extraction method or the alkaline extraction method.

FIG. 5 shows the % area of the purified 18B spider silk monomer and impurities after tangential flow filtration (TFF) as measured by SEC.

FIG. 6A shows the total yield of 18B spider silk protein after a two-step extraction. Results from two different runs are shown. FIG. 6B shows the 18B purity as measured by SEC percent area after a two-step extraction.

FIG. 7A shows % area of 18B monomer, low (LMW), and intermediate molecular weight (IMW) impurities after alkaline extraction of whole cell broth. The extracted protein was concentrated using tangential flow filtration. FIG. 7B shows the SEC analysis of recovered 18B spider silk protein. The 18B monomer peak of the various tangential flow filtration fractions are indicated by the arrows.

FIG. 8 shows the % area of 18B monomer, high (HMW), low (LMW), and intermediate molecular weight (IMW) impurities after alkaline extraction and pH precipitation of whole cell broth. The extracted protein was concentrated using diacentrifugation.

FIG. 9 shows the % yield of 18B monomer after alkaline extraction and pH precipitation of whole cell broth. The extracted protein was concentrated using diacentrifugation.

FIG. 10 shows the SEC analysis of purified 18B spider silk protein after acid precipitation at pH 6. The 18B monomer peak is indicated by the arrow. The extracted protein was concentrated using diacentrifugation.

FIG. 11 shows an immunoblot of soluble PO protein after extraction from E. coli lysate using various pH buffers or urea.

### DETAILED DESCRIPTION OF THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and polypeptide and nucleic acid chemistry and hybridization described herein are those well-known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

The terms “fermenting” and “fermentation” as used herein describe culturing host cells under conditions to produce a desired product, including but not limited to conditions under which the host cells grow.

The term “fermentation broth” as used herein refers to an aqueous medium used to culture host cells during fermentation.

The term “inoculum” as used herein refers to a quantity of host cells that are added to a fermentation broth to start a fermentation.

The term “clarifying” as use herein refers to a method removing host cell biomass, such as whole cells, lysed cells, membranes, lipids, organelles, nuclei, non-spider silk proteins, or any other undesirable cell part or product, or any other undesirable portion of a cell culture. Clarifying may also refer to removing impurities from a partially purified or isolated spider silk composition. Impurities may include, but are not limited to, non-spider silk proteins, degraded spider silk proteins, large aggregates of proteins, chemicals used during the purification and isolation process, or any other undesirable material.

The term “purity” as used herein refers to the amount of full-length isolated recombinant spider silk protein as a portion of all isolated components, such as partial or degraded isolated recombinant spider silk proteins, lipids, proteins, membranes, or other molecules in a sample, such as an extracted sample.

The term “yield” as used herein refers to the amount of full-length recombinant spider silk protein isolated from a cell culture compared to the amount of full-length silk protein or total silk protein in a control sample. The percentage can be in reference to the total amount of full length spider silk protein in a cell lysate, a crude alkaline extraction solution, a partially purified or filtered alkaline extraction solution, a purified solution subject to alkaline extraction methods or a purified solution subject to control extraction methods such as urea or GdSCN. as described herein.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

The term “recombinant” refers to a biomolecule, e.g., a gene or polypeptide, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as polypeptides and/or mRNAs encoded by such nucleic acids.

As used herein, an endogenous nucleic acid sequence in the genome of an organism (or the encoded polypeptide product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it. In an embodiment, a heterologous nucleic acid molecule is not endogenous to the organism. In further embodiments, a heterologous nucleic acid molecule is a plasmid or molecule integrated into a host chromosome by homologous or random integration.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “percent sequence identity” in the context of nucleic acid sequences refers to the quantitative value of an alignment of the residues in the two sequences when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wisc. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Nucleic acids (also referred to as polynucleotides) can include both sense and antisense strands of RNA, cDNA, genomic DNA, and synthetic forms and mixed polymers of the above. They can be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

The term “expression system” as used herein includes vehicles or vectors for the expression of a gene in a host cell as well as vehicles or vectors which bring about stable integration of a gene into the host chromosome.

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance polypeptide stability; and when desired, sequences that enhance polypeptide secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “promoter,” as used herein, refers to a DNA region to which RNA polymerase binds to initiate gene transcription, and positions at the 5′ direction of an mRNA transcription initiation site.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “polypeptide” encompasses both naturally-occurring and non-naturally- occurring proteins, and fragments, mutants, derivatives and analogs thereof. A polypeptide may be monomeric or polymeric. Further, a polypeptide may comprise a number of different domains each of which has one or more distinct activities.

As used herein, the term “molecule” means any compound, including, but not limited to, a small molecule, peptide, polypeptide, sugar, nucleotide, nucleic acid, polynucleotide, lipid, etc., and such a compound can be natural or synthetic.

The term “block” or “repeat unit” as used herein refers to a subsequence greater than approximately 12 amino acids of a natural silk polypeptide that is found, possibly with modest variations, repeatedly in said natural silk polypeptide sequence and serves as a basic repeating unit in said silk polypeptide sequence. Blocks may, but do not necessarily, include very short “motifs.” A “moti” as used herein refers to an approximately 2-10 amino acid sequence that appears in multiple blocks. For example, a motif may consist of the amino acid sequence GGA, GPG, or AAAAA. A sequence of a plurality of blocks is a “block co-polymer.”

As used herein, the term “repeat domain” refers to a sequence selected from the set of contiguous (unbroken by a substantial non-repetitive domain, excluding known silk spacer elements) repetitive segments in a silk polypeptide. Native silk sequences generally contain one repeat domain. In some embodiments of the present invention, there is one repeat domain per silk molecule. A “macro-repeat” as used herein is a naturally occurring repetitive amino acid sequence comprising more than one block. In an embodiment, a macro-repeat is repeated at least twice in a repeat domain. In a further embodiment, the two repetitions are imperfect. A “quasi-repeat” as used herein is an amino acid sequence comprising more than one block, such that the blocks are similar but not identical in amino acid sequence.

A “repeat sequence” or “R” as used herein refers to a repetitive amino acid sequence. In an embodiment, a repeat sequence includes a macro-repeat or a fragment of a macro-repeat. In another embodiment, a repeat sequence includes a block. In a further embodiment, a single block is split across two repeat sequences.

It must be noted that, as used in the specification and the appended claims, the singular forms “a,” “an” and “the” include plural referents unless the context clearly dictates otherwise.

Any ranges disclosed herein are inclusive of the extremes of the range. For example, a range of 2-5% includes 2% and 5%, and any number or fraction of a number in between, for example: 2.25%, 2.5%, 2.75%, 3%, 3.25%, 3.5%, 3.75%, 4%, 4.25%, 4.5%, and 4.75%.

### Recombinant Spider Silk Compositions

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSpl and MaSp2. MaSpl silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent silk-like fibers that recapitulate the properties of corresponding natural silk fibers.

### Silk Nucleotide and Peptide Sequences

A list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for microbial expression, for example in Pichia (Komagataella) pastoris or Escherichia coli. The DNA sequences are each cloned into an expression vector and transformed into a microbe such as Pichia (Komagataella) pastoris or Escherichia coli. In some embodiments, various silk domains demonstrating successful expression and secretion are subsequently assembled in combinatorial fashion to build silk molecules capable of fiber formation.

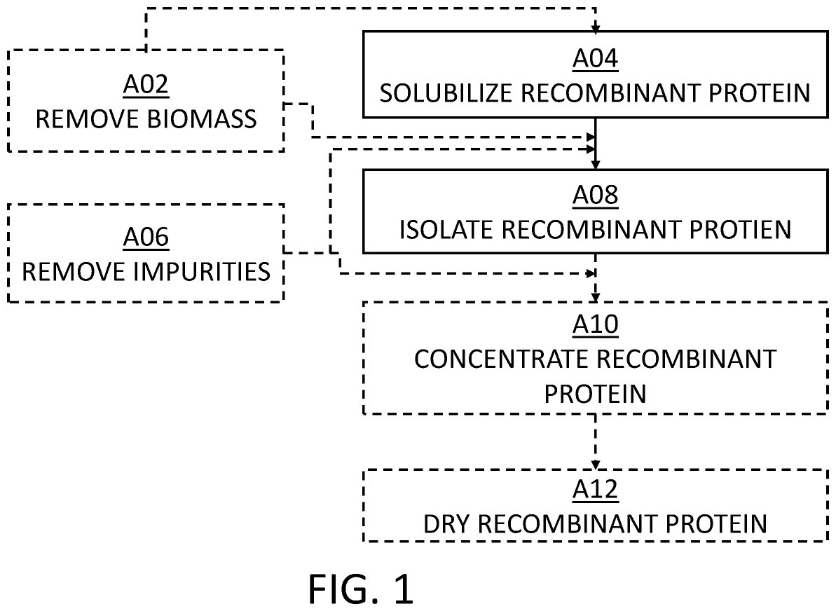

Silk polypeptides are characteristically composed of a repeat domain (REP) flanked by non-repetitive regions (e.g., C-terminal and N-terminal domains). The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. Block sequences may comprise a glycine rich region followed by a polyA region. Short (˜1-10) amino acid motifs may appear multiple times inside of blocks. A subset of commonly observed motifs is depicted in FIG. 1. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG is, for the purposes of the present invention, the same as GSGAG and the same as GGSGA; they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1
Samples of Block Sequences

| | Silk | SEQ | |
|---|---|---|---|
| Species | Type | ID NO | Representative Block Amino Acid Sequence |
| Aliatypus | Fibroin | 1 | GAASSSSTIITTKSASASAAADASAAATASAASRSSANAAASAFAQSF |
| gulosus | 1 | | SSILLESGYFCSIFGSSISSSYAAAIASAASRAAAESNGYTTHAYACA |
| | | | KAVASAVERVTSGADAYAYAQAISDALSHALLYTGRLNTANANSLASA |
| | | | FAYAFANAAAQASASSASAGAASASGAASASGAGSAS |
| Plectreurys | Fibroin | 2 | GAGAGAGAGAGAGAGAGSGASTSVSTSSSSGSGAGAGAGSGAGSGAGA |
| tristis | 1 | | GSGAGAGAGAGGAGAGFGSGLGLGYGVGLSSAQAQAQAQAAAQAQAQA |
| | | | QAQAYAAAQAQAQAQAQAQAAAAAAAAAAA |
| Plectreurys | Fibroin | 3 | GAAQKQPSGESSVATASAAATSVTSGGAPVGKPGVPAPIFYPQGPLQQ |
| tristis | 4 | | GPAPGPSNVQPGTSQQGPIGGVGGSNAFSSSFASALSLNRGFTEVISS |
| | | | ASATAVASAFQKGLAPYGTAFALSAASAAADAYNSIGSGANAFAYAQA |
| | | | FARVLYPLVQQYGLSSSAKASAFASAIASSFSSGTSGQGPSIGQQQPP |
| | | | VTISAASASAGASAAAVGGGQVGQGPYGGQQQSTAASASAAAATATS |
| Araneus | TuSp | 4 | GNVGYQLGLKVANSLGLGNAQALASSLSQAVSAVGVGASSNAYANAVS |
| gemmoides | | | NAVGQVLAGQGILNAANAGSLASSFASALSSSAASVASQSASQSQAAS |
| | | | QSQAAASAFRQAASQSASQSDSRAGSQSSTKTTSTSTSGSQADSRSAS |
| | | | SSASQASASAFAQQSSASLSSSSSFSSAFSSATSISAV |
| Argiope | TuSp | 5 | GSLASSFASALSASAASVASSAAAQAASQSQAAASAFSRAASQSASQS |
| aurantia | | | AARSGAQSISTTTTTSTAGSQAASQSASSAASQASASSFARASSASLA |
| | | | ASSSFSSAFSSANSLSALGNVGYQLGFNVANNLGIGNAAGLGNALSQA |
| | | | VSSVGVGASSSTYANAVSNAVGQFLAGQGILNAANA |
| Deinopis | TuSp | 6 | GASASAYASAISNAVGPYLYGLGLFNQANAASFASSFASAVSSAVASA |
| spinosa | | | SASAASSAYAQSAAAQAQAASSAFSQAAAQSAAAASAGASAGAGASAG |
| | | | AGAVAGAGAVAGAGAVAGASAAAASQAAASSSASAVASAFAQSASYAL |
| | | | ASSSAFANAFASATSAGYLGSLAYQLGLTTAYNLGLSNAQAFASTLSQ |
| | | | AVTGVGL |
| Nephila | TuSp | 7 | GATAASYGNALSTAAAQFFATAGLLNAGNASALASSFARAFSASAESQ |
| clavipes | | | SFAQSQAFQQASAFQQAASRSASQSAAEAGSTSSSTTTTTSAARSQAA |
| | | | SQSASSSYSSAFAQAASSSLATSSALSRAFSSVSSASAASSLAYSIGL |
| | | | SAARSLGIADAAGLAGVLARAAGALGQ |
| Argiope | Flag | 8 | GGAPGGGPGGAGPGGAGFGPGGGAGFGPGGGAGFGPGGAAGGPGGPGG |
| trifasciata | | | PGGPGGAGGYGPGGAGGYGPGGVGPGGAGGYGPGGAGGYGPGGSGPGG |
| | | | AGPGGAGGEGPVTVDVDVTVGPEGVGGGPGGAGPGGAGFGPGGGAGFG |
| | | | PGGAPGAPGGPGGPGGPGGPGGPGGVGPGGAGGYGPGGAGGVGPAGTG |
| | | | GFGPGGAGGFGPGGAGGFGPGGAGGFGPAGAGGYGPGGVGPGGAGGFG |
| | | | PGGVGPGGSGPGGAGGEGPVTVDVDVSV |
| Nephila | Flag | 9 | GVSYGPGGAGGPYGPGGPYGPGGEGPGGAGGPYGPGGVGPGGSGPGGY |
| clavipes | | | GPGGAGPGGYGPGGSGPGGYGPGGSGPGGYGPGGSGPGGYGPGGSGPG |
| | | | GYGPGGYGPGGSGPGGSGPGGSGPGGYGPGGTGPGGSGPGGYGPGGSG |
| | | | PGGSGPGGYGPGGSGPGGFGPGGSGPGGYGPGGSGPGGAGPGGVGPGG |
| | | | FGPGGAGPGGAAPGGAGPGGAGPGGAGPGGAGPGGAGPGGAGPGGAGG |
| | | | AGGAGGSGGAGGSGGTTIIEDLDITIDGADGPITISEELPISGAGGSG |
| | | | PGGAGPGGVGPGGSGPGGVGPGGSGPGGVGPGGSGPGGVGPGGAGGPY |
| | | | GPGGSGPGGAGGAGGPGGAYGPGGSYGPGGSGGPGGAGGPYGPGGEGP |
| | | | GGAGGPYGPGGAGGPYGPGGAGGPYGPGGEGGPYGP |
| Latrodectus | AcSp | 10 | GINVDSDIGSVTSLILSGSTLQMTIPAGGDDLSGGYPGGFPAGAQPSG |
| hesperus | | | GAPVDFGGPSAGGDVAAKLARSLASTLASSGVFRAAFNSRVSTPVAVQ |
| | | | LTDALVQKIASNLGLDYATASKLRKASQAVSKVRMGSDTNAYALAISS |
| | | | ALAEVLSSSGKVADANINQIAPQLASGIVLGVSTTAPQFGVDLSSINV |
| | | | NLDISNVARNMQASIQGGPAPITAEGPDFGAGYPGGAPTDLSGLDMGA |
| | | | PSDGSRGGDATAKLLQALVPALLKSDVFRAIYKRGTRKQVVQYVTNSA |
| | | | LQQAASSLGLDASTISQLQTKATQALSSVSADSDSTAYAKAFGLAIAQ |
| | | | VLGTSGQVNDANVNQIGAKLATGILRGSSAVAPRLGIDLS |
| Argiope | AcSp | 11 | GAGYTGPSGPSTGPSGYPGPLGGGAPFGQSGFGGSAGPQGGFGATGGA |
| trifasciata | | | SAGLISRVANALANTSTLRTVLRTGVSQQIASSVVQRAAQSLASTLGV |
| | | | DGNNLARFAVQAVSRLPAGSDTSAYAQAFSSALFNAGVLNASNIDTLG |
| | | | SRVLSALLNGVSSAAQGLGINVDSGSVQSDISSSSSFLSTSSSSASYS |
| | | | QASASSTS |
| Uloborus | AcSp | 12 | GASAADIATAIAASVATSLQSNGVLTASNVSQLSNQLASYVSSGLSST |
| diversus | | | ASSLGIQLGASLGAGFGASAGLSASTDISSSVEATSASTLSSSASSTS |
| | | | vvssINAQLVPALAQTAVLNAAFSNINTQNAIRIAELLTQQVGRQYGL |
| | | | SGSDVATASSQIRSALYSVQQGSASSAYVSAIVGPLITALSSRGVVNA |
| | | | SNSSQIASSLATAILQFTANVAPQFGISIPTSAVQSDLSTISQSLTAI |
| | | | SSQTSSSVDSSTSAFGGISGPSGPSPYGPQPSGPTFGPGPSLSGLTGF |
| | | | TATFASSFKSTLASSTQFQLIAQSNLDVQTRSSLISKVLINALSSLGI |
| | | | SASVASSIAASSSQSLLSVSA |
| Euprosthenops | MaSp1 | 13 | GGQGGQGQGRYGQGAGSSAAAAA |
| australis | | | |
| Tetragnatha | MaSp1 | 14 | GGLGGGQGAGQGGQQGAGQGGYGSGLGGAGQGASAAAAAAAA |
| kauaiensis | | | |
| Argiope | MaSp2 | 15 | GGYGPGAGQQGPGSQGPGSGGQQGPGGLGPYGPSAAAAAAAA |
| aurantia | | | |
| Deinopis | MaSp2 | 16 | GPGGYGGPGQQGPGQGQYGPGTGQQGQGPSGQQGPAGAAAAAAAAA |
| spinosa | | | |
| Nephila | MaSp2 | 17 | GPGGYGLGQQGPGQQGPGQQGPAGYGPSGLSGPGGAAAAAAA |
| clavata | | | |
| Deinopis | MiSp | 18 | GAGYGAGAGAGGGAGAGTGYGGGAGYGTGSGAGYGAGVGYGAGAGAGG |
| Spinosa | | | GAGAGAGGGTGAGAGGGAGAGYGAGTGYGAGAGAGGGAGAGAGAGAGA |
| | | | GAGAGSGAGAGYGAGAGYGAGAGAGGVAGAGAAGGAGAAGGAGAAGGA |
| | | | GAAGGAGAGAGAGSGAGAGAGGGARAGAGG |
| Latrodectus | MiSp | 19 | GGGYGRGQGAGAGVGAGAGAAAGAAAIARAGGYGQGAGGYGQGQGAGA |
| hesperus | | | AAGAAAGAGAGGYGQGAGGYGRGQGAGAGAGAGAGARGYGQGAGAGAA |
| | | | AGAAASAGAGGYGQGAGGYGQGQGAGAAAGAAASAGAGGYGQGAGGYG |
| | | | QGQGA |
| Nephila | MiSp | 20 | GAGAGGAGYGRGAGAGAGAAAGAGAGAAAGAGAGAGGYGGQGGYGAGA |
| clavipes | | | GAGAAAAAGAGAGGAAGYSRGGRAGAAGAGAGAAAGAGAGAGGYGGQG |
| | | | GYGAGAGAGAAAAAGAGSGGAGGYGRGAGAGAAAGAGAAAGAGAGAGG |
| | | | YGGQGGYGAGAGAAAAA |
| Nephilengys | MiSp | 21 | GAGAGVGGAGGYGSGAGAGAGAGAGAASGAAAGAAAGAGAGGAGGYGT |
| cruentata | | | GQGYGAGAGAGAGAGAGGAGGYGRGAGAGAGAGAGGAGGYGAGQGYGA |
| | | | GAGAGAAAAAGDGAGAGGAGGYGRGAGAGAGAGAAAGAGAGGAGGYGA |
| | | | GQGYGAGAGAGAAAGAGAGGAGGYGAGQGYGAGAGAGAAAAA |
| Uloborus | MiSp | 22 | GSGAGAGSGYGAGAGAGAGSGYGAGSSASAGSAINTQTVTSSTTTSSQ |
| diversus | | | SSAAATGAGYGTGAGTGASAGAAASGAGAGYGGQAGYGQGAGASARAA |
| | | | GSGYGAGAGAAAAAGSGYGAGAGAGAGSGYGAGAAA |
| Uloborus | MiSp | 23 | GAGAGYRGQAGYIQGAGASAGAAAAGAGVGYGGQAGYGQGAGASAGAA |
| diversus | | | AAAGAGAGRQAGYGQGAGASAGAAAAGAGAGRQAGYGQGAGASAGAAA |
| | | | AGADAGYGGQAGYGQGAGASAGAAASGAGAGYGGQAGYGQGAGASAGA |
| | | | AAAGAGAGYLGQAGYGQGAGASAGAAAGAGAGYGGQAGYGQGTGAAAS |
| | | | AAASSA |
| Araneus | MaSp1 | 24 | GGQGGQGGYGGLGSQGAGQGGYGAGQGAAAAAAAAGGAGGAGRGGLGA |
| ventricosus | | | GGAGQGYGAGLGGQGGAGQAAAAAAAGGAGGARQGGLGAGGAGQGYGA |
| | | | GLGGQGGAGQGGAAAAAAAAGGQGGQGGYGGLGSQGAGQGGYGAGQGG |
| | | | AAAAAAAAGGQGGQGGYGGLGSQGAGQGGYGGRQGGAGAAAAAAAA |
| Dolomedes | MaSp1 | 25 | GGAGAGQGSYGGQGGYGQGGAGAATATAAAAGGAGSGQGGYGGQGGLG |
| tenebrosus | | | GYGQGAGAGAAAAAAAAAGGAGAGQGGYGGQGGQGGYGQGAGAGAAAA |
| | | | AAGGAGAGQGGYGGQGGYGQGGGAGAAAAAAAASGGSGSGQGGYGGQG |
| | | | GLGGYGQGAGAGAGAAASAAAA |
| Nephdengys | MaSp | 26 | GGAGQGGYGGLGGQGAGAAAAAAGGAGQGGYGGQGAGQGAAAAAASGA |
| cruentata | | | GQGGYEGPGAGQGAGAAAAAAGGAGQGGYGGLGGQGAGQGAGAAAAAA |
| | | | GGAGQGGYGGLGGQGAGQGAGAAAAAAGGAGQGGYGGQGAGQGAAAAA |
| | | | AGGAGQGGYGGLGSGQGGYGRQGAGAAAAAAAA |
| Nephdengys | MaSp | 27 | GGAGQGGYGGLGGQGAGAAAAAAGGAGQGGYGGQGAGQGAAAAAASGA |
| cruentata | | | GQGGYGGPGAGQGAGAAAAAAGGAGQGGYGGLGGQGAGQGAGAAAAAA |
| | | | GGAGQGGYGGQGAGQGAAAAAAGGAGQGGYGGLGSGQGGYGGQGAGAA |
| | | | AAAGGAGQGGYGGLGGQGAGQGAGAAAAAA |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a Glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3X FLAG nucleotide coding sequence. In some embodiments, the silk polypeptide nucleotide coding sequence is operatively linked to other affinity tags such as 6-8 His residues.

### Secretion Signals

The amount of protein that is secreted from a cell varies significantly between proteins, and is dependent in part on the secretion signal that is operably linked to the protein in its nascent state. A number of secretion signals are known in the art, and some are commonly used for production of secreted recombinant proteins, including microbial secretion signals of Pichia pastoris and Saccharomyces cerevisiae. Prominent among these is the secretion signal of the α-mating factor (αMF) of Saccharomyces cerevisiae, which consists of a N-terminal 19-amino-acid signal peptide (also referred to herein as pre-αMF(sc)) followed by a 70-amino-acid leader peptide (also referred to herein as pro-αMF(sc)). Inclusion of pro-αMF(sc) in the secretion signal of the αMF of Saccharomyces cerevisiae (also referred to herein as pre-αMF(sc)/pro-αMF(sc) has proven critical for achieving high secreted yields of proteins. Addition of pro-αMF(sc) or functional variants thereof to signal peptides other than pre-αMF(sc) has also been explored as a means of achieving secretion of recombinant proteins, but has shown variable degrees of effectiveness, increasing secretion for certain recombinant proteins in certain recombinant host cells but having no effect or decreasing secretion for other recombinant proteins.

The use of multiple distinct secretion signals can improve the secreted yields of recombinant proteins produced in host cells such as P. pastoris, as described in U.S. application Ser. No. 15/724,196. Compared to recombinant host cells that comprise multiple polynucleotide sequences encoding a recombinant protein operably linked to just one secretion signal (e.g., pre-αMF(sc)/pro-αMF(sc)), recombinant host cells that comprise the same number of polynucleotide sequences encoding the recombinant protein operably linked to at least 2 distinct secretion signals produce higher secreted yields of the recombinant protein. Without wishing to be bound by theory, the use of at least 2 distinct secretion signals may permit the recombinant host cell to engage distinct cellular secretory pathways to effect efficient secretion of the recombinant protein and thus prevent over-saturation of any one secretion pathway.

At least one of the distinct secretion signals comprises a signal peptide may be selected from Table 2 or 3 or is a functional variant that has an at least 80% amino acid sequence identity to a signal peptide selected from Table 2 or 3. In some embodiments, the functional variant is a signal peptide selected from Table 2 or 3 that comprises one or two substituted amino acids. In some such embodiments, the functional variant has an at least 85%, at least 90%, at least 95%, or at least 99% amino acid sequence identity to a signal peptide selected from Table 2 or 3. In some embodiments, the signal peptide mediates translocation of the nascent recombinant protein into the ER post-translationally (i.e., protein synthesis precedes translocation such that the nascent recombinant protein is present in the cell cytosol prior to translocating into the ER). In other embodiments, the signal peptide mediates translocation of the nascent recombinant protein into the ER co-translationally (i.e., protein synthesis and translocation into the ER occur simultaneously). An advantage of using a signal peptide that mediates co-translational translocation into the ER is that recombinant proteins prone to rapid folding are prevented from assuming conformations that hinder translocation into the ER and thus secretion.

TABLE 2
Secretion Signals

| Source Gene | | | SEQ | |
|---|---|---|---|---|
| ID | Species | Name | ID NO | Sequence |
| PEP4 | Saccharomyces | pre- | 28 | MFSLKALLPLALLLVSANQVAA |
| | cerevisiae | PEP4(sc) | | |
| PAS_chr1- | Pichia pastoris | pre- | 29 | MSFSSNVPQLFLLLVLLTNIVSG |
| 1_0130 | | DSE4(pp) | | |
| PAS_chr3_ | Pichia pastoris | pre- | 30 | MKLSTNLILAIAAASAVVSA |
| 0076 | | EPX1(pp) | | |
| P00698 | Gallus gallus | pre | 31 | MRSLLILVLCFLPLAALG |
| | | CLSP(gg) | | |

TABLE 3
Recombinant Secretion Signals

| | SEQ ID | |
|---|---|---|
| Name | NO | Sequence |
| pre-αMF(sc)/ | 32 | MRFPSIFTAVLFAASSALAAPVNTTTEDETAQIPAEAVIGYL |
| pro-αMF(sc) | | DLEGDFDVAVLPFSNSTNNGLLFINTTIASIAAKEEGVSLDK |
| | | REAEA |
| pre-αMF(sc)/ | 33 | MRFPSIFTAVLFAASSALAAPVNTTTEDETAQIPAEAVIGYS |
| *pro-αMF(sc) | | DLEGDFDVAVLPFSNSTNNGLLFINTTIASIAAKEEGVSLEK |
| | | REAEA |
| pre-PEP4(sc)/ | 34 | MFSLKALLPLALLLVSANQVAAAPVNTTTEDETAQIPAEAVI |
| *pro-aMF(sc) | | GYSDLEGDFDVAVLPFSNSTNNGLLFINTTIASIAAKEEGVS |
| | | LEKREAEA |
| pre-DSE4(pp)/ | 35 | MSFSSNVPQLFLLLVLLTNIVSGAPVNTTTEDETAQIPAEAV |
| *pro-αMF(sc) | | IGYSDLEGDFDVAVLPFSNSTNNGLLFINTTIASIAAKEEGV |
| | | SLEKREAEA |
| pre-EPX1(pp)/ | 36 | MKLSTNLILAIAAASAVVSAAPVNTTTEDETAQIPAEAVIGY |
| *pro-αMF(sc) | | SDLEGDFDVAVLPFSNSTNNGLLFINTTIASIAAKEEGVSLE |
| | | KREAEA |
| pre-CLSP(gg)/ | 37 | MRSLLILVLCFLPLAALGAPVNTTTEDETAQIPAEAVIGYSD |
| *pro-αMF(sc) | | LEGDFDVAVLPFSNSTNNGLLFINTTIASIAAKEEGVSLEKR |
| | | EAEA |

### Expression Vectors

The expression vectors of the present invention can be produced following the teachings of the present specification in view of techniques known in the art. Sequences, for example vector sequences or sequences encoding transgenes, can be commercially obtained from companies such as Integrated DNA Technologies, Coralville, Iowa or Atum, Menlo Park, Calif. Exemplified herein are expression vectors that direct high-level expression of the chimeric silk polypeptides.

Another standard source for the polynucleotides used in the invention is polynucleotides isolated from an organism (e.g., bacteria), a cell, or selected tissue. Nucleic acids from the selected source can be isolated by standard procedures, which typically include successive phenol and phenol/chloroform extractions followed by ethanol precipitation. After precipitation, the polynucleotides can be treated with a restriction endonuclease which cleaves the nucleic acid molecules into fragments. Fragments of the selected size can be separated by a number of techniques, including agarose or polyacrylamide gel electrophoresis or pulse field gel electrophoresis (Care et al. (1984) Nuc. Acid Res. 12:5647-5664; Chu et al. (1986) Science 234:1582; Smith et al. (1987) Methods in Enzymology 151:461), to provide an appropriate size starting material for cloning.

Another method of obtaining the nucleotide components of the expression vectors or constructs is PCR. General procedures for PCR are taught in MacPherson et al., PCR: A PRACTICAL APPROACH, (IRL Press at Oxford University Press, (1991)). PCR conditions for each application reaction may be empirically determined. A number of parameters influence the success of a reaction. Among these parameters are annealing temperature and time, extension time, Mg2+ and ATP concentration, pH, and the relative concentration of primers, templates and deoxyribonucleotides. Exemplary primers are described below in the Examples. After amplification, the resulting fragments can be detected by agarose gel electrophoresis followed by visualization with ethidium bromide staining and ultraviolet illumination.

Another method for obtaining polynucleotides is by enzymatic digestion. For example, nucleotide sequences can be generated by digestion of appropriate vectors with suitable recognition restriction enzymes. Restriction cleaved fragments may be blunt ended by treating with the large fragment of E. coli DNA polymerase I (Klenow) in the presence of the four deoxynucleotide triphosphates (dNTPs) using standard techniques.

Polynucleotides are inserted into suitable backbones, for example, plasmids, using methods well known in the art. For example, insert and vector DNA can be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers can contain nucleic acid sequences that correspond to a particular restriction site in the vector DNA. Other means are known and available in the art. A variety of sources can be used for the component polynucleotides.

In some embodiments, expression vectors containing an R, N, or C sequence are transformed into a host organism for expression and secretion. In some embodiments, the expression vectors comprise a secretion signal. In some embodiments, the expression vector comprises a terminator signal. In some embodiments, the expression vector is designed to integrate into a host cell genome and comprises: regions of homology to the target genome, a promoter, a secretion signal, a tag (e.g., a FLAG tag), a termination/polyA signal, a selectable marker for Pichia, a selectable marker for E. coli, an origin of replication for E. coli, and restriction sites to release fragments of interest.

### Host Cell Transformants

Host cells transformed with nucleic acid molecules or vectors that express spider silk polypeptides, and descendants thereof, are provided. These cells can also carry the nucleic acid sequences of the present invention on vectors, which may but need not be freely replicating vectors. In other embodiments of the present invention, the nucleic acids have been integrated into the genome of the host cells.

In some embodiments, microorganisms or host cells that enable the large-scale production of block copolymer polypeptides of the invention include a combination of: 1) the ability to produce large (>40 kDa) polypeptides, 2) the ability to secrete polypeptides outside of the cell and circumvent costly downstream intracellular purification, 3) resistance to contaminants (such as viruses and bacterial contaminations) at large-scale, and/or 4) the existing know-how for growing and processing the organism is large-scale (1-2000 m3) bioreactors.

A variety of host organisms can be engineered/transformed to comprise a block copolymer polypeptide expression system. Preferred organisms for expression of a recombinant silk polypeptide include yeast, fungi, gram-negative, and gram-positive bacteria. In certain embodiments, the host organism is Arxula adeninivorans, Aspergillus aculeatus, Aspergillus awamori, Aspergillus ficuum, Aspergillus fumigatus, Aspergillus japonicus, Aspergillus nidulans, Aspergillus niger, Aspergillus oryzae, Aspergillus sojae, Aspergillus tubigensis, Bacillus alkalophilus, Bacillus amyloliquefaciens, Bacillus anthracis, Bacillus brevis, Bacillus circulans, Bacillus coagulans, Bacillus lautus, Bacillus lentus, Bacillus licheniformis, Bacillus methanolicus, Bacillus stearothermophilus, Bacillus subtilis, Bacillus thuringiensis, Candida boidinii, Chrysosporium lucknowense, Escherichia coli, Fusarium graminearum, Fusarium venenatum, Kluyveromyces lactis, Kluyveromyces marxianus, Myceliopthora thermophila , Neurospora crassa, Ogataea polymorpha, Penicillium camemberti, Penicillium canescens, Penicillium chrysogenum, Penicillium emersonii, Penicillium funiculosum, Penicillium griseoroseum, Penicillium purpurogenum, Penicillium roqueforti, Phanerochaete chrysosporium, Pichia angusta, Pichia methanolica, Pichia (Komagataella) pastoris, Pichia polymorpha, Pichia stipitis, Rhizomucor miehei, Rhizomucor pusillus, Rhizopus arrhizus, Streptomyces lividans, Saccharomyces cerevisiae, Schwanniomyces occidentalis, Trichoderma harzianum, Trichoderma reesei, or Yarrowia lipolytica.

In preferred aspects, the methods provide culturing host cells for direct product secretion for easy recovery without the need to extract biomass. In some embodiments, the block copolymer polypeptides are secreted directly into the medium for collection and processing.

### Engineered Host Cell Lines

Any appropriate host cell line can be used to produce recombinant proteins. The methylotrophic yeast Pichia pastoris is widely used in the production of recombinant proteins. P. pastoris grows to high cell density, provides tightly controlled methanol-inducible trans gene expression and efficiently secretes heterologous proteins in defined media. However, during culture of a strain of P. pastoris, recombinantly expressed proteins may be degraded before they can be collected, resulting in a mixture of proteins that includes fragments of recombinantly expressed proteins and a decreased yield of full-length recombinant proteins. Another widely used cell line for recombinant protein production is the bacteria Escherichia coli.

In some embodiments, the modified strains with reduced protease activity described herein recombinantly express a silk-like polypeptide sequence. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful fibers by secretion from an industrially scalable microorganism. Large (approximately 40 kDa to approximately 100 kDa) block copolymer polypeptides engineered from silk repeat domain fragments, including sequences from almost all published amino acid sequences of spider silk polypeptides, can be expressed in the modified microorganisms described herein. In some embodiments, silk polypeptide sequences are matched and designed to produce highly expressed and secreted polypeptides capable of fiber formation. In some embodiments, knock-out of protease genes or reduction of protease activity in the host modified strain reduces degradation of the silk like polypeptides.

In some embodiments, to attenuate a protease activity in Pichia pastoris, the genes encoding these enzymes are inactivated or mutated to reduce or eliminate activity. This can be done through mutations or insertions into the gene itself of through modification of a gene regulatory element. This can be achieved through standard yeast genetics techniques. Examples of such techniques include gene replacement through double homologous recombination, in which homologous regions flanking the gene to be inactivated are cloned in a vector flanking a selectable maker gene (such as an antibiotic resistance gene or a gene complementing an auxotrophy of the yeast strain).

Alternatively, the homologous regions can be PCR-amplified and linked through overlapping PCR to the selectable marker gene. Subsequently, such DNA fragments are transformed into Pichia pastoris through methods known in the art, e.g., electroporation. Transformants that then grow under selective conditions are analyzed for the gene disruption event through standard techniques, e.g. PCR on genomic DNA or Southern blot. In an alternative experiment, gene inactivation can be achieved through single homologous recombination, in which case, e.g. the 5′ end of the gene's ORF is cloned on a promoterless vector also containing a selectable marker gene. Upon linearization of such vector through digestion with a restriction enzyme only cutting the vector in the target-gene homologous fragment, such vector is transformed into Pichia pastoris. Integration at the target gene site is confirmed through PCR on genomic DNA or Southern blot. In this way, a duplication of the gene fragment cloned on the vector is achieved in the genome, resulting in two copies of the target gene locus: a first copy in which the ORF is incomplete, thus resulting in the expression (if at all) of a shortened, inactive protein, and a second copy which has no promoter to drive transcription.

Alternatively, transposon mutagenesis is used to inactivate the target gene. A library of such mutants can be screened through PCR for insertion events in the target gene.

The functional phenotype (i.e., deficiencies) of an engineered/knockout strain can be assessed using techniques known in the art. For example, a deficiency of an engineered strain in protease activity can be ascertained using any of a variety of methods known in the art, such as an assay of hydrolytic activity of chromogenic protease substrates, band shifts of substrate proteins for the selected protease, among others.

Attenuation of a protease activity described herein can be achieved through mechanisms other than a knockout mutation. For example, a desired protease can be attenuated via amino acid sequence changes by altering the nucleic acid sequence, placing the gene under the control of a less active promoter, down-regulation, expressing interfering RNA, ribozymes or antisense sequences that target the gene of interest, or through any other technique known in the art. In preferred strains, the protease activity of proteases encoded at PAS_chr4_0584 (YPS1-1) and PAS_chr3_1157 (YPSI-2) is attenuated by any of the methods described above. In some aspects, the invention is directed to methylotrophic yeast strains, especially Pichia pastoris strains, wherein a YPS1-1 and a YPS1-2 gene have been inactivated. In some embodiments, additional protease encoding genes may also be knocked-out in accordance with the methods provided herein to further reduce protease activity of a desired protein product expressed by the strain.

In some embodiments, the P. pastoris strains disclosed herein have been modified to express a silk-like polypeptide. Methods of manufacturing preferred embodiments of silk-like polypeptides are provided in WO 2015/042164, especially at Paragraphs 114-134, incorporated herein by reference. Disclosed therein are synthetic proteinaceous copolymers based on recombinant spider silk protein fragment sequences derived from MaSp2, such as from the species Argiope bruennichi. Silk-like polypeptides are described that include two to twenty repeat units, in which a molecular weight of each repeat unit is greater than about 20 kDa. Within each repeat unit of the copolymer are more than about 60 amino acid residues that are organized into a number of “quasi-repeat units.” In some embodiments, the repeat unit of a polypeptide described in this disclosure has at least 95% sequence identity to a MaSp2 dragline silk protein sequence.

### Methods for Producing and Purifying a Recombinant Protein

The methods provided herein comprise fermenting an inoculum of a recombinant host cell provided herein in a suitable fermentation broth and a suitable fermentation vessel under suitable fermentation conditions for production of a desired cumulative yield and/or cumulative titer and/or cumulative productivity of a recombinant protein.

In some embodiments, the recombinant host cell secretes the recombinant protein. In various embodiments, the recombinant host cell can be a prokaryote that does not secrete the recombinant protein. In a specific embodiment, the recombinant host cell is Escherichia coli.

In various embodiments, the recombinant host cell can be a eukaryote that secretes the recombinant protein or a prokaryote such as a gram-negative or gram-positive bacteria that secretes the recombinant protein. In some embodiments, the recombinant host cell is Pichia pastoris. In specific embodiments, the recombinant host cell is a Pichia pastoris strain with activity of one or more proteases abrogated (e.g. by functional knock out). In addition, specific embodiments discussed below are applicable to the production of recombinant hydrophobic or partially-hydrophobic proteins, such as silk proteins.

U.S. Pat. No. 9,963,554, “Methods and Compositions for Synthesizing Improved Silk Fibers,” incorporated herein by reference, discloses compositions for synthetic block copolymers, recombinant microorganisms for their production, and synthetic fibers comprising the proteins. U.S. patent application Ser. No. 15/724,196, “Modified Strains for the Production of Recombinant Silk,” incorporated herein by reference, discloses engineered Pichia pastoris cells selected or genetically engineered to reduce degradation of recombinant proteins expressed by the yeast cells, and to methods of cultivating yeast cells for the production of useful compounds. Other appropriate microbial strains, including Escherichia coli, can be cultivated and used in the production of useful compounds.

### Fermentation

In some embodiments, the inoculum of the recombinant host cell can be derived from a seed train (i.e., series of fermentations of increasing volume to generate an adequate number of recombinant host cells). Depending on the embodiment, the number of seeds may range from 2-7, 3-7, 3-6, or 3-5 seeds.

In some embodiments, the inoculum of the recombinant host cell has a dry cell weight (DCW) per liter of media of at least 0.2 g/L, at least 0.5 g/L, at least 0.7 g/L, at least 0.8 g/L, at least 1 g/L, at least 2 g/L, at least 3 g/L, at least 4 g/L, or at least 5g/L; between 0.2 g/L and 3 g/L, 0.2 g/L and 2 g/L, or 0.2 g/L and 1 g/L; between 0.5 g/L and 3 g/L, 0.5 g/L and 2 g/L, or 0.5 g/L and 1 g/L; between 1 g/L and 3 g/L, 1 g/L and 2 g/L, or 0.5 g/L and 1 g/L; or between 3 g/L and 1 g/L. DCW can be measured using a biophotometer (e.g., an Eppendorf Bio Photometer D30).

In most embodiments, the size of the inoculum will depend on the size of the fermentation vessel. In embodiments where the size of the fermentation vessel is less than 150L the DCW can range from 0.1 g/L-0.5 g/L. In embodiments where the size of the fermentation vessel is greater than 150L, the DCW can range from 2-4 g/L.

Depending on the specific embodiment, a suitable fermentation broth is any fermentation broth in which the recombinant host cell can subsist (i.e. maintain growth and/or viability). Non-limiting examples of suitable fermentation broths include aqueous media comprising nutrients required for growth and/or viability of the recombinant host cell. Non-limiting examples of such nutrients include carbon sources, nitrogen sources, phosphate sources, salts, minerals, bases, acids, vitamins (e.g., biotin), amino acids, and metals (e.g., iron, zinc, calcium, copper, sodium, potassium, cobalt, magnesium, manganese).

In some embodiments, any of the above nutrients may be limited in order to inhibit cell growth and improve the productivity, yield or titer of recombinant proteins. The carbon source can be any carbon source that can be fermented by the recombinant host cell. Non-limiting examples of suitable carbon sources include monosaccharides, disaccharides, polysaccharides, acetate, ethanol, methanol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, and combinations thereof

The nitrogen source can be any nitrogen source that can be assimilated (i.e., metabolized) by the recombinant host cell. Non-limiting examples of suitable nitrogen sources include anhydrous ammonia, ammonium sulfate, ammonium nitrate, diammonium phosphate, monoammonium phosphate, ammonium polyphosphate, sodium nitrate, urea, peptone, protein hydrolysates, yeast extract and any of the above enriched with air or oxygen.

In some embodiments, any or all of the nutrients can be sterilized using heat or ozonation in order to reduce or eliminate microbial contamination before addition to the fermentation broth. For example, the carbon source can be caramelized or sterilized using heat before addition to the fermentation broth. Similarly, carbon sources may be ozonated before addition to the fermentation broth. Suitable methods for ozonation are discussed in Dziugan et al., Ozonation as an effective way to stabilize new kinds of fermentation media used in biotechnological production of liquid fuel additives, Biotechnology for Biofuels, 9:150 (2016).

The fermentation broth can comprise an acid or a base to adjust and/or maintain a pH. In some such embodiments, the pH is between 4.0 and 8.0, 7.5, 7.0, 6.5, 6.0, 5.5, 5.0, or 4.5; between 4.5 and 8.0, 7.5, 7.0, 6.5, 6.0, 5.5, or 5.0; between 5.0 and 8.0, 7.5, 7.0, 6.5, 6.0, or 5.5; between 5.5 and 8.0, 7.5, 7.0, 6.5, or 6.0; between 6.0 and 8.0, 7.5, 7.0, or 6.5; between 6.5 and 8.0, 7.5, or 7.0; between 7.0 and 8.0, or 7.5; or between 7.5 and 8.0.

Non-limiting examples of suitable acids include aspartic acid, acetic acid, hydrochloric acid, and sulfuric acid. Non-limiting examples of suitable bases include sodium hydroxide, potassium hydroxide, calcium hydroxide, ammonium hydroxide, calcium carbonate, ammonia, and diammonium phosphate. In some embodiments, strong acids or strong bases are used to limit dilution of the fermentation broth.

In some embodiments, the fermentation broth comprises such nutrients or such amounts of such nutrients that a desired oxygen uptake rate (OUR) is achieved and/or maintained. In some such embodiments, the desired OUR is at least 40 mmol O₂/L/hr, at least 80 mmol O₂/L/hr, at least 100 mmol O₂/L/hr, at least 105 mmol O₂/L/h, at least 110 mmol O₂/L/h, at least 115 mmol O₂/L/h, at least 120 mmol O₂/L/hr, or at least 140 mmol O₂/L/hr, at least 160 mmol O₂/L/hr, at least 180 mmol O₂/L/hr, at least 200 mmol O₂/L/hr, or at least 220 mmol O₂/L/hr; between 40 mmol O₂/L/hr and 220 mmol O₂/L/hr, 60 mmol O₂/L/hr and 220 mmol O₂/L/hr, 80 mmol O₂/L/hr and 220 mmol O₂/L/hr, or 100 mmol O₂/L/hr and 220 mmol O₂/L/hr; between 100 mmol O₂/L/hr and 140 mmol O₂/L/hr, 100 mmol O₂/L/hr and 135 mmol O₂/L/hr, 100 mmol O₂/L/hr and 130 mmol O₂/L/hr, or 100 mmol O₂/L/hr and 125 mmol O₂/L/hr; between 110 mmol O₂/L/hr and 125 mmol O₂/L/hr, or 110 mmol O₂/L/hr and 120 mmol O₂/L/hr; or between 115 mmol O₂/L/hr and 120 mmol O₂/L/hr. The OUR can be calculated by one of ordinary skill in the art using the Direct Method described in Bioreaction Engineering Principles 3^{rd} Edition, 2011, Spring Science+Business Media, p. 449.

In some embodiments, the fermentation broth comprises such nutrients or such amounts of such nutrients that production of the recombinant protein by the recombinant host cell is increased in relation to production of byproducts. Non-limiting examples of such byproducts include ethanol. In some embodiments, in 72 hours of fermentation the recombinant host cells produce ethanol at a cumulative yield of less than 0.1 g/L, less than 1 g/L, less than 5 g/L, less than 10 g/L, or less than 15g/L; between 0.1 g/L and 15 g/L, 1 g/L and 15 g/L, 5 g/L and 15 g/L, 10 g/L and 15 g/L, or 0.5 g/L and 15 g/L; or between 0.1 g/L and 1.5 g/L, 0.2 g/L and 1.5 g/L, 0.5 g/L and 1.5 g/L, 0.7 g/L and 1.5 g/L or 1.0 g/L and 1.5 g/L.

In some embodiments, the fermentation broth comprises such nutrients or such amounts of such nutrients that a desired dissolved oxygen (DO) content is reached and/or maintained. In some such embodiments, the desired DO content is at least 2%, 5%, 10%, 20%, 30%, 40%, 50%, 60%, 70%, 80% or 100%; or between 2% and 40%, 2% and 5%, 5% and 40%, 2% and 20%, 5% and 20%, 2 and 15%, or 5% and 15%.

In some embodiments, the fermentation broth comprises such nutrients or such amounts of such nutrients that a desired respiratory quotient (RQ; i.e., the ratio of carbon dioxide produced to oxygen consumed) is reached and/or maintained. In In some such embodiments, the desired RQ is less than 2, less than 1.75, less than 1.5, or less than 1.25; or between 1 and 1.1, 1 and 1.2, 1 and 1.3, 1 and 1.4, or 1 and 1.5.

In some embodiments, the fermentation broth comprises such nutrients or such amounts of such nutrients that a desired doubling time of the recombinant host cell is reached and/or maintained. In some such embodiments, the desired doubling time is at least 4 hours, 8 hours, 12 hours, 16 hours, 18 hours, 22 hours, 26 hours, 30 hours, 34 hours or 36 hours; or between 4 hours and 12 hours, 4 hours and 10 hours, 4 hours and 8 hours, 6 hours and 12 hours, 6 hours and 10 hours, or 6 hours and 8 hours.

In some embodiments, the fermentation broth comprises one or more supplemental proteins. Addition of such supplemental proteins can serve to distract protease activity from the recombinant protein produced by the recombinant host cell in embodiments where the recombinant host cell secretes the recombinant protein. Non-limiting examples of supplemental proteins include: bovine serum albumin (BSA) and cassamino acids. Other supplemental proteins are well known in the art.

The nutrients can be added to the fermentation broth either in a one-time bolus, incrementally, or continuously. In embodiments in which the nutrients are added continuously, they can be added at fast, slow, or exponential speed.

In embodiments, where the nutrients are continuous added to the fermentation broth, the nutrients may be added by the continuous addition of medium containing the nutrients. In these embodiments, an equal volume of aqueous media in the fermentation broth may be removed from the fermentation so that the total volume of the fermentation broth remains the same. In some embodiments, recombinant host cells may be removed from the fermentation broth and re-added to the medium containing the nutrients before addition to the fermentation broth.

The suitable fermentation vessel is any fermentation vessel in which the recombinant host cell can subsist (maintain growth and/or viability). Non-limiting examples of suitable fermentation vessels include a culture plate, a vial, a flask, or a fermentor. Non-limiting examples of suitable fermentors include a stirred tank fermentor, an airlift fermentor, a bubble column reactor, a fixed bed bioreactor, and any combination thereof

The suitable fermentation conditions are any conditions under which the recombinant host cell can subsist (maintain growth and/or viability). Non-limiting examples of such fermentation conditions include a suitable volume of fermentation broth, a suitable pH of the fermentation broth, a suitable DO in the fermentation broth, a suitable temperature, a suitable oxygenation, a suitable agitation of the recombinant host cell and a suitable duration of fermenting.

In various embodiments, suitable temperature can be any temperature suitable for growth and/or viability of the recombinant host cells and/or production of recombinant protein. In some embodiments, the temperature is at least 15° C., 20° C., 25° C., 30° C., 35° C.; between 15° C. to 35° C., 15° C. to 25° C., 15° C. to 20° C., 20° C. to 35° C., 20° C. to 30° C., 20° C. to 25° C., 25° C. to 35° C. or 25° C. to 30° C.

A suitable oxygenation can be any oxygenation suitable for growth and/or viability of the recombinant host cells and/or production of the recombinant host cell. Such oxygenation can be achieved by providing a suitable aeration and/or a suitable agitation of the fermentation vessel and/or fermentation broth. In some embodiments, the suitable aeration is at least 1.5 vvm, at least 1.6 vvm, at least 1.7 vvm, at least 1.8 vvm, at least 1.9 vvm, or at least 2 vvm; between 1.5 vvm and 2 vvm, 1.5 vvm and 1.9 vvm, 1.5 vvm and 1.8 vvm, 1.5 vvm and 1.7 vvm, 1.5 vvm and 1.6 vvm, 1.6 vm and 2 vvm, 1.7 vvm and 2 vvm, 1.8 vvm and 2 vvm, or 1.7 vvm and 1.9 vvm.

Depending on the embodiment and the type of fermentation, a suitable agitation of the recombinant host cell in the fermentation broth can vary.

Depending on the embodiment, a bubble column may be used for aeration. Bubble columns may vary in complexity based on the specific embodiment (e.g. may be single or multiple phase) and may provide various gas velocities. Non-limiting examples of suitable gas velocities include but are not limited to 0.003-0.08 m/s. Non-limiting examples of bubble reactors are included in Kantarci et al., Bubble Column Reactors, Process Biochemistry 40:2263-2283 (2005).

In some embodiments, the fermentation broth comprises an agent to reduce foam during fermentation (“antifoam agent”). Foam, as defined herein, is the dispersion of gas in the continuous liquid phase located in or near the top of the fermentation vessel. Depending on the embodiment, the anti-foaming agent may be selected and optimized to reduce interaction with any recombinant protein product. Non-limiting examples of anti-foaming agents include silicon-based oils, emulsions and polymers; polypropylene glycol; polyethylene glycol-based antifoam agents; polyalkylene glycol-based antifoam agents; difunctional ethylene/propylene oxide (EO/PO) block copolymers; fatty acid-based antifoam agents; polyester-based antifoam agents oil-based antifoam agents and any combination of the foregoing. Suitable antifoam agents are discussed in Junker, Foam and its Mitigation in Fermentation Systems, Biotechnol. Prog., 23:767-784 (2007). In embodiments where the recombinant protein is a hydrophobic protein, such as a silk protein, the antifoam agent may be selected so that it solubilizes or does not solubilize the hydrophobic protein.

The desired cumulative yield of the recombinant protein can be any cumulative yield that contributes to low production cost. As used herein, cumulative yield is calculated as the percentage of the mass of the recombinant protein produced of the mass of carbon source catabolized by the recombinant host cell over the course of the fermenting (i.e., mass of carbon source provided minus mass of carbon source remaining in the fermentation broth; for example, if 100 grams of glucose are provided to the recombinant host cell, and at the end of fermenting 25 grams of the recombinant protein are produced and there remains 10 grams of glucose, the cumulative yield of the recombinant protein is 27.7%). Assuming all other metrics are equal, a higher cumulative yield provides lower production cost than a lower cumulative yield. In some embodiments, the cumulative yield of the recombinant silk protein on carbon source after 72 hours of fermenting is at least 1%, at least 5%, at least 30%, or at least 100%; between 1% and 5%, between 5% and 10%, between 10% and 35%, between 35% and 50%, or between 50% and 100%.

The desired cumulative titer of the recombinant protein can be any cumulative titer that contributes to low production cost. As used herein, cumulative titer is calculated as grams of recombinant protein produced per liter of fermentation broth over the course of the fermenting (i.e., g/L). Assuming all other metrics are equal, a higher cumulative titer provides lower production cost than a lower cumulative titer. In some embodiments, the cumulative titer of the recombinant protein after 72 hours of fermentation is at least 2 g/L, at least 5 g/L, at least 15 g/L, or at least 30 g/L; between 1 g/L and 100 g/L, 5 g/L, 15 g/L, or 30g/L; between 10 g/L and 100 g/L, 80 g/L, or 75g/L; or between 5 g/L and 30 g/L.

The desired cumulative productivity of the recombinant protein can be any cumulative productivity that contributes to low production cost. As used herein, cumulative productivity is calculated as grams of recombinant protein produced per liter of fermentation broth per hour over the course of the fermenting (i.e., g/L/hr). Assuming all other metrics are equal, a higher cumulative productivity provides lower production cost than a lower cumulative productivity. In some embodiments, the cumulative productivity of the recombinant protein is at least 0.001 g/L/hr, at least 0.025 g/L/hr, at least 0.05 g/L/hr, at least 0.1 g/L/hr, or at least 0.2g/L/hr; between 0.001 g/L/hr and 0.5 g/L/hr.

The methods provided herein can be performed at any fermentation scale and/or according to any fermentation procedure known in the art. The fermentation procedures can be fed-batch, batch, continuous, or any combination thereof. In some embodiments, the methods commence with one or more batch fermentations followed by one or more continuous fermentations, wherein the inoculum of the recombinant host cell, the suitable fermentation broth, the suitable fermentation vessel, and/or one or more of the suitable fermentation conditions can differ between the one or more batch fermentations and/or the one or more continuous fermentation. In some embodiments, the temperature of the batch fermentation is higher than the temperature of the continuous fermentation. In some such embodiments, the temperature of the batch fermentation is more than 27° C., and the temperature of the continuous fermentation is less than 27° C.

In some embodiments, the fermenting proceeds in phases. Such phases may comprise a growth phase, a production phase, and/or a recovery phase. In some embodiments, the phases differ from each other in the inoculum of the recombinant host cell, the suitable fermentation broth, the suitable fermentation vessel, and/or one or more of the suitable fermentation conditions.

### Methods for Isolating a Recombinant Protein

Depending on the embodiment, various methods may be used to isolate and recover the recombinant protein of interest. As discussed above, some, but not all, of these methods are specific to recombinant host cells that secrete the recombinant protein of interest. Further, some of these methods are specific to recombinant proteins of interest that are hydrophobic.

FIG. 1 depicts a process flow for isolating a recombinant protein according to one embodiment of the present invention. Persons who are skilled in the art will understand that some of the steps illustrated in FIG. 1 can be performed in an alternate order and/or in repetition. Persons skilled in the art will recognize that the disclosed embodiments are not intended to limit the scope of the methods provided herein, and that the methods may be varied based on the recombinant host cell used, the desired cumulative yield, cumulative titer, and/or cumulative productivity, or other factors.

At optional step A02, biomass (i.e. intact or disrupted recombinant host cells and cell debris) is removed from the fermentation comprising recombinant host cells. In various embodiments, removing biomass can also include removing insoluble fermentation impurities (such as, for example, antifoaming agents and other components of the fermentation broth that may have precipitated during the solubilizing of the protein).

In various embodiments, removing biomass can be accomplished based on size, weight, density, or a combination thereof. Removing biomass based on size can be accomplished via filtration using, for example, a filter press, candlestick filter, or other industrially used filtration system with a molecular weight cutoff that is smaller than the size of the recombinant host cells. Removing biomass based on weight or density can be accomplished via gravitational settling or centrifugation, using, for example, a settler, low g-force decanter centrifuge, disk stack separator, 2-phase nozzle centrifuge, solids ejector centrifuge, or hydrocyclone. Removing biomass as disclosed herein yields a centrate (i.e., light phase or clear cell broth) that comprises the protein, and solids (heavy phase) comprising the biomass and insoluble fermentation impurities. Suitable conditions for removing biomass (e.g., g-forces, settling time, centrifugation time, % solids in centrifuge input, centrifuge feed rate) can be determined using methods known in the art geared towards minimizing biomass and insoluble fermentation impurities in the clear cell broth. In some embodiments, removing biomass provides a clear cell broth that has a wet packed solids volume of less than 5%, less than 1%, less than 0.5% or less than 0.1%. In some embodiments, removing biomass provides a clear cell broth that comprises protein at a concentration of between 1 g/L and 50 g/L. In some embodiments, the clear cell broth is subjected to a polishing centrifugation to remove remaining solids. In some embodiments, the solids obtained from removing biomass are subjected to at least one more round of solubilizing the protein and removing biomass, wherein all centrates are finally combined for further processing according to the methods provided herein.

Depending on the embodiment, step A02 may be performed before and/or after step A04. Step A02 may be performed several times. For example, several rounds of centrifugation and/or filtration may be performed to remove biomass before and/or after step A04.

At step A04, the recombinant protein is solubilized. In some embodiments where step A02 is not performed, the recombinant protein may be isolated along with the recombinant host cells prior to solubilization by centrifuging the recombinant host cells and recombinant protein associated with the recombinant host cells into a pellet of biomass (hereinafter “cell pellet”) and discarding the supernatant. This step may be beneficial in instances where the recombinant protein is insoluble and/or aggregates with itself and/or with the recombinant host cells and/or sticks to the surface of the recombinant host cells. In other embodiments, the recombinant protein is solubilized in a whole cell broth. In some embodiments, the recombinant protein is solubilized in a clear cell broth generated by performing step A02.

In some embodiments, solubilizing the recombinant protein can be accomplished by adding a solubilization agent to the whole cell broth, clear cell broth or cell pellet. Non limiting examples of suitable solubilization agents include surfactants, hydrotropes, SDS, urea, cysteine, guanidine thiocyanate, enzymes that hydrolyze polysaccharides (e.g., glucanase, lyticase, mannase, chitinase), high pH water (H₂O at a pH of 11-12), or other known chaotropes. Different solubilization agents may be selected for different types of recombinant proteins. Suitable conditions for solubilizing proteins (e.g., type and amount of extraction agent, temperature, incubation time, agitation, pH) can be determined using methods known in the art geared towards maximizing the yield of the recombinant protein, and minimizing lysis of the recombinant host cells and solubilizing of impurities. As discussed above, in a specific embodiment where the recombinant protein is insoluble and/or aggregates with itself and/or in or near the recombinant host cells, the recombinant host cells may be centrifuged and the supernatant may be discarded before adding the solubilization agent to the pellet.

In some embodiments, various techniques may be used to perforate or permeabilize the membrane of the recombinant host cell in order to remove excess protein from the membrane of prior to solubilization and/or precipitation. Such methods include chemical disruption, mechanical disruption, or sonication. Mechanical disruption of cell membranes includes homogenization, shear force, freeze/thawing, heating, pressure, sonication, and filtration. Chemical disruption includes detergents such as triton, sodium dodecyl sulfate; or chaotropic agents such as urea and guanidine. Other methods are well known in the art.

In a specific embodiment, urea is used to as a solubilization agent to solubilize the recombinant protein and prevent disruption of the recombinant host cells. The concentration of urea may be varied to prevent disruption of the recombinant host cells. Depending on the embodiment and the amount of the concentration of urea may range from 4M to 10M. In various embodiments, the recombinant host cells and recombinant protein may be incubated with urea for 1-2 hours, 1-3 hours, or 1-4 hours. Depending on the embodiment, other known chaotropes such as guanidine thiocyante are used to solubilize the recombinant protein.

In a specific embodiment, high pH H₂O or aqueous buffer is used to solubilize the recombinant protein and prevent disruption of the recombinant host cells. The pH of the high pH H₂O or aqueous buffer may be varied to prevent disruption of the recombinant host cells. Depending on the embodiment, the pH of the high pH H₂O may range from pH 10 to pH 12.5, pH 10.5 to pH 12.5, pH 11 to pH 12.5, pH 11.5 to pH 12.5, pH 12 to pH 12.5, pH 10 to pH 12, pH 10.5 to pH 11.0, pH 10.5 to pH 11.5, pH 10.5 to pH 12, pH 10.5 to pH 12.5, pH 11 to pH 11.5, pH 11 to pH 12, pH 11.5 to pH 12.5, or ph 12 to pH 12.5. In various embodiments, the recombinant host cells and recombinant protein may be incubated with high pH H₂O for at least 10 minutes, at least 15 minutes, at least 30 minutes, at least 45 minutes, at least 60 minutes, at least 75 minutes, at least 90 minutes, at least 115 minutes or at least 120 minutes.

In a specific embodiment, homogenization is used to lyse the host cell. Homogenization pressure (psi) may be between 5,000-100,000 psi, 5,000-10,000 psi, 10,000-20,000 psi, 20,000-30,000 psi, 30,000-40,000 psi, 40,000-50,000 psi, 50,000-60,000 psi, 60,000-70,000 psi, 70,000-80,000 psi, 80,000-90,000 psi, 90,000-100,000 psi. Homogenization may be a single pass or multiple passes. In some embodiments, the homogenization is one pass, two passes, three passes, four passes, or five passes.

At step A06, impurities are removed from the fermentation. Step A06 may be performed before and/or after step A04 and/or step A08. Step A06 may be repeated any number of times. Removing impurities from the fermentation can be accomplished by filtration, absorption (e.g. charcoal or solid-state absorption), dialysis and phase separation induced by coacervation or the use of various chemicals. In embodiments where phase separation is induced by coacervation, coacervation may induced by chilling the fermentation to a temperature sufficient to induce phase separation. In other embodiments, phase separation may be chemically induced by adding a cosmotrope and/or a compound used to precipitate the protein from solution. A detailed embodiment of impurity removal using phase separation is described below with respect to Figure C. In some embodiments, where the recombinant protein is heat stable, other proteins may be removed by subjecting the fermentation to high temperatures to denature the other proteins and centrifugation to separate the denatured proteins from the proteins in solution.

In some embodiments, impurities are removed using filtration, microfiltration, diafiltration and/or ultrafiltration (e.g., against deionized water). Membranes suitable for microfiltration may include 0.1 uM to 1 uM. Non-limiting examples of suitable membranes for ultrafiltration include hydrophobic membranes (e.g., PES, PS, cellulose acetate) with molecular weight cut-offs of between 50 kDa and 800 kDa, 100 kDa and 800 kDa, 200 kDa and 800 kDa, 300 kDa and 800 kDa, 400 kDa and 800 kDa, 500 kDa and 800 kDa, 600 kDa and 800 kDa, 700 kDa and 800 kDa, 100 kDa and 700 kDa, 200 kDa and 700 kDa, 300 kDa and 700 kDa, 400 kDa and 700 kDa, 500 kDa and 700 kDa, 600 kDa and 700 kDa, or 500 kDa and 600 kDa. In some embodiments, ultrafiltration yields as retentate a recombinant protein slurry in water, and a permeate comprising the impurities. Suitable conditions for ultrafiltration (e.g., membranes, temperature, volume replacement) can be determined using methods known in the art geared towards maximizing permeate density. In some embodiments, the ultrafiltration provides a rententate that has a density of between 1 g/mL and 30 g/mL. In some embodiments, ultrafiltration comprises a concentrating step that yields a concentrated retentate, followed by a diafiltration step that removes the impurities and yields the suspended protein slurry in water. In some such embodiments, the concentrated retentate has a concentration factor of between 2-fold and 12-fold volume reduction to starting volume. In some embodiments, the diafiltration provides a constant volume replacement of between 3-fold and 10-fold.

Depending on the embodiment and the type of impurity to be removed, methods of removing impurities may differ. Removing lipid impurities from the isolated recombinant protein can be accomplished by methods known in the art. Non-limiting examples of such methods include absorption to charcoals or other absorption media that specifically bind lipids. Removing polysaccharide impurities from the isolated recombinant protein can be accomplished by methods known in the art. Non-limiting examples of such methods include treatment with enzymes that hydrolyze polysaccharides followed by removal of the small sugars produced by ultrafiltration. Non-limiting examples of such enzymes include glucanase, lyticase, mannase, and chitinase.

At step A08, the solubilized recombinant protein is isolated. The solubilized recombinant protein can be isolated in a number of different ways including using an extraction buffer, size exclusion chromatography, gel filtration, ultrasonic protein extraction and ion exchange chromatography. In some embodiments where the biomass is not removed at optional step A02, the recombinant protein may be isolated along with the recombinant host cells.

In some embodiments, the recombinant protein is precipitated as a single isolation step or in addition to other isolation steps. Precipitating the solubilized recombinant protein can be accomplished by adding to fermentation a precipitation agent. Non-limiting examples of such precipitation agents include sulfate ions (e.g. Ammonium Sulfate, Sodium Sulfate, Sulfuric acid) or citrate ions (e.g. Sodium Citrate). In some embodiments, the precipitating agent is an acid. In some embodiments, the precipitating agent is a salt. In one embodiment, the precipitating agent is H₂SO₄.

Any appropriate acid may be used to adjust or alter the pH of the solution comprising the solubilized recombinant protein. Appropriate acids include mineral acids such as hydrochloric acid (HCl), sulfuric acid (H2SO4), nitric acid, (HNo3), boric acid (H3BO3), phorsphroic acid (H3PO4), hydrofluoric acid (HF), hydrobromic acid (HBr), perchloric acid (HClO4), hydroiodic acid (HI); organic acids such as citric acid, formic acid, acetic acid, propionic acid, butyric acid, valeric acid, caprioc acid, oxalic acid, lactic acid, malic acid, benzoic acid, carbonic acid, uric acid, taurine, p-toluenesulfonic acid, trifluoromethanesulfonic acid, aminomethylphophonic acid, and 2,2,2,-trichloroacetic acid (TCA); or any combination thereof or other appropriate acid known in the art. Acid salts of any of the acids disclosed above may also be used.

In some embodiments, the recombinant protein is precipitated at pH 4-10. In some embodiments, the precipitation is at pH 4, 5, 6, 7, 8, 9, or 10. In some embodiments, the precipitation is at least pH 4, at least pH 4.5, at least pH 5, at least pH 5.5, at least pH 6, at least pH 6.5, at least pH 7, at least pH 7.5, at least pH 8, at least pH 8.5, at least pH 9, at least pH 9.5, at least pH 10. In one embodiment, the precipitation is at pH 7. In some embodiments, the precipitation is from pH 4-5, pH 5-6, pH 6-7, pH 7-8, pH 8-9, or pH 9-10.

The precipitation may be repeated once, twice, or as many times as required. In some embodiments, more than one precipitation step is performed and the pH of each precipitation is the same. In other embodiments, more than one precipitation step is performed and the pH of each precipitation is different. For example, the first precipitation may be performed at pH 4, and then a second precipitation may be performed at pH 7.

Isolating the precipitated recombinant protein can be accomplished based on size, weight, density, or a combination thereof, as disclosed herein. In some embodiments, such isolating provides as retentate a suspended recombinant protein slurry, and a permeate comprising waste. Suitable conditions for precipitating the recombinant protein (e.g., dilution prior to addition of divalent anion, type and amount of divalent anion, incubation temperature, incubation time) and isolating the precipitated recombinant protein can be determined using methods known in the art geared towards maximizing the yield of recombinant protein in the suspended recombinant protein slurry. In some embodiments, the yield of the precipitated recombinant protein in the suspended silk protein slurry is between 20% and 99%. In some embodiments, the suspended silk protein slurry has a wet packed solids content of between 30% and 65%. In some embodiments, the suspended silk protein slurry comprises the silk protein at a concentration of between 10 g/L and 50 g/L. In some embodiments, the steps of precipitating the silk protein and isolating the precipitated silk protein are repeated at least once (using identical or different process conditions) to further wash away aqueous soluble impurities.

At optional step A10, the isolated recombinant protein is concentrated. Concentrating the isolated recombinant protein can be accomplished by evaporation at elevated temperature and/or reduced pressure (e.g., partial vacuum). Suitable conditions (e.g., temperature, pressure, duration) for concentrating the isolated recombinant protein can be determined using methods known in the art geared towards obtaining an isolated recombinant protein with increased content of dry solids. In some embodiments, the concentrating provides a reduction in volume of between 20% and 70% of the original volume. In some embodiments, the concentrating provides a concentrated isolated recombinant protein that comprises between 3% and 20% of dry solids.

At optional step A12, the isolated recombinant protein is dried. Drying of the suspended silk protein slurry to obtain a silk protein powder can be accomplished by spray drying, drum dryers, lyophilization, or fluid bed drying. In some embodiments, the powder has a moisture content of less than 10%, less than 9%, less than 8%, less than 6%, less than 5%, less than 4%, less than 3%, less than 2% or less than 1%.

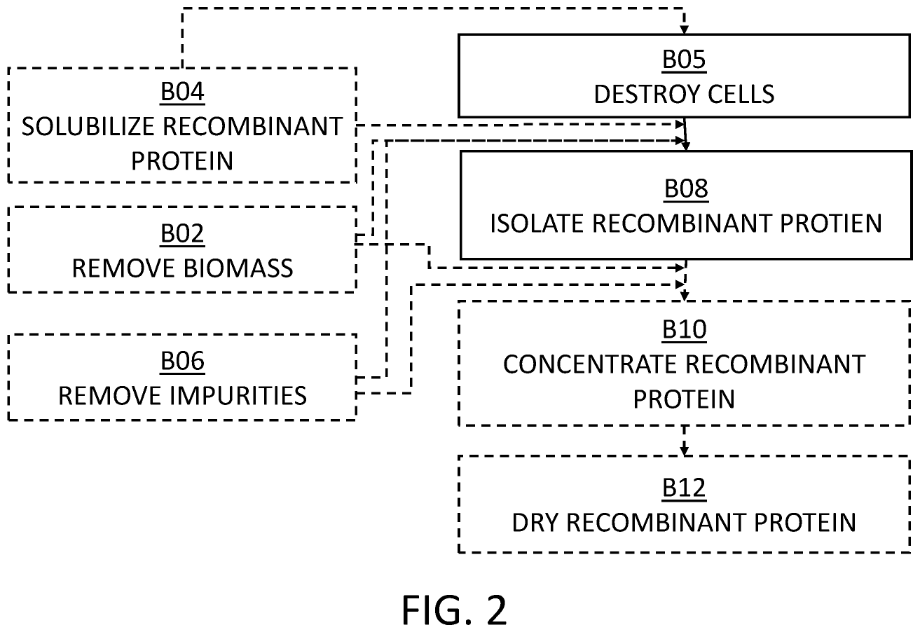

FIG. 2 depicts a process flow for isolating a recombinant protein according to one embodiment of the present invention. Persons who are skilled in the art will understand that some of the steps illustrated in FIG. 2 can be performed in an alternate order and/or in repetition. Persons skilled in the art will recognize that the disclosed embodiments are not intended to limit the scope of the methods provided herein, and that the methods may be varied based on the recombinant host cell used, the desired cumulative yield, cumulative titer, and/or cumulative productivity, or other factors.

At step B05, the recombinant host cells are lysed and/or otherwise disrupted so that the contents of the recombinant host cells are released into the fermentation. Depending on the embodiment, recombinant host cells may be destroyed using a variety of different methods. Suitable methods for lysing and/or disrupting host cells include: using heat such as High Temperature Short Time (HTST) methods, high shear cell disruption, physical homogenization and chemical homogenization.

At optional step B04, the recombinant protein is solubilized as described above with respect to step A04. Step B04 may be performed before or after step B05. In some embodiments, step B04 may be performed before and after step B05.

At optional step B02, the biomass is removed as described above with respect to step A02. In addition, other methods of removing biomass from the lysed and/or disrupted cells can include centrifugation and filtration in instances where the recombinant protein is solubilized.

At optional step B06, the impurities are removed as described above with respect to step A06. Steps B02 and B06 may be performed before or after other steps and performed in repetition. In some embodiments, step B06 may be performed before and after step B08.

At step B08, the recombinant protein is isolated. Suitable methods for isolating the recombinant protein are described above with respect to step A08. In addition, methods for isolating the recombinant protein can also include using additional membranes in filtration and/or degumming to remove phospholipids.

At optional step B10, the recombinant protein is concentrated as described above with respect to step A10. At optional step B12, the recombinant protein is dried as described above with respect to step B10.

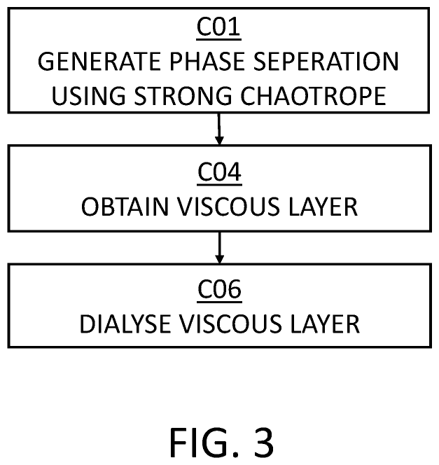

FIG. 3 depicts a process flow for recombinant protein purification according to one embodiment of the present invention. Persons who are skilled in the art will understand that some of the steps illustrated in FIG. 3 can be performed in an alternate order and/or in repetition. Persons skilled in the art will recognize that the disclosed embodiments are not intended to limit the scope of the methods provided herein, and that the methods may be varied based on the various factors.

At step CO2, an aqueous two-phase solution is created using a strong chaotrope to denature the recombinant protein. Suitable chaotropes include but are not limited to: guanidine thiocyanate (GD-SCN), guanidine hydrochloride (GD-HCl), guanidine iodide, urea, lithium perchlorate, lithium acetate, magnesium chloride, sodium dodecyl sulfate (SDS), potassium iodide (KI) or any combination thereof. Depending on the embodiment, the chaotrope and protein may be heated to facilitate denaturation of the protein.

In some embodiments, a kosmotrope (also referred to herein as a “precipitation agent”) is added to the solution to facilitate phase-separation. Suitable kosmotropes include the precipitation agents referenced above. In other embodiments, a high starting concentration of the chaotrope is used to denature the recombinant protein, then the concentration of the chaotrope slowly diluted in order to obtain phase separation.

At step C04, the viscous layer of the phase separation is obtained. Depending on the type of phase separation various methods may be used to obtain the viscous layer such as decanting/extracting the non-viscous layer or using Hamilton needles or pipettes to extract the viscous layer. Other methods will be known to those skilled in the art.

As step C06, the viscous layer of the phase separation is further processed to remove impurities. Suitable dialysis agents include double distilled H2O, or GD-SCN at a low concentration. Depending on the embodiment, various methods of dialysis may be performed include cassette dialysis or other suitable methods known in the art. In some embodiments, tangential flow filtration (TFF) is used to dialyze the viscous layer.

In some embodiments, the isolated recombinant spider silk protein is at least 60%, at least 65%, at least 70%, at least 75%, at least 80%, at least 85%, at least 90%, at least 95%, or at least 99% full-length recombinant spider silk protein.

In some embodiments, the purity of the isolated recombinant spider silk protein is 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 90-95%, or 95-100%. In some embodiments, the purity of the isolated recombinant spider silk protein is at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In some embodiments, the full-length recombinant spider silk protein is measured or quantified. Any appropriate method may be used to measure or quantify the amount of full length recombinant protein, including, but not limited so, size exclusion chromatography (SEC), SDS-PAGE, immunoblot (Western blot), high performance liquid chromatography (HPLC), liquid chromatography-mass spectrometry (LC-MS), or fast protein liquid chromatography (FPLC), or any other appropriate method known in the art, or any combination thereof. In one embodiment, the amount of full-length recombinant spider silk protein is measured using a western blot. In another embodiment, the amount of full-length recombinant spider silk protein is measured using size exclusion chromatography (SEC).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B(1992).

### Example 1

### 18B Purification Using One-Step Alkaline Conditions

High pH solutions were used to solubilize recombinant proteins without disrupting the host cells that secrete the recombinant protein. pH buffer solution concentration and incubation times were tested to determine solubility for a recombinant spider silk protein from Argiope bruennichi MaSp2 blocks (“18B,” SEQ ID NO: 38) with a C terminal 3x FLAG tag (SEQ ID NO: 40) that was expressed in P. Pastoris. The FLAG tag is linked at the C terminus of the 18B peptide sequence with a glycine residue (G) linker.

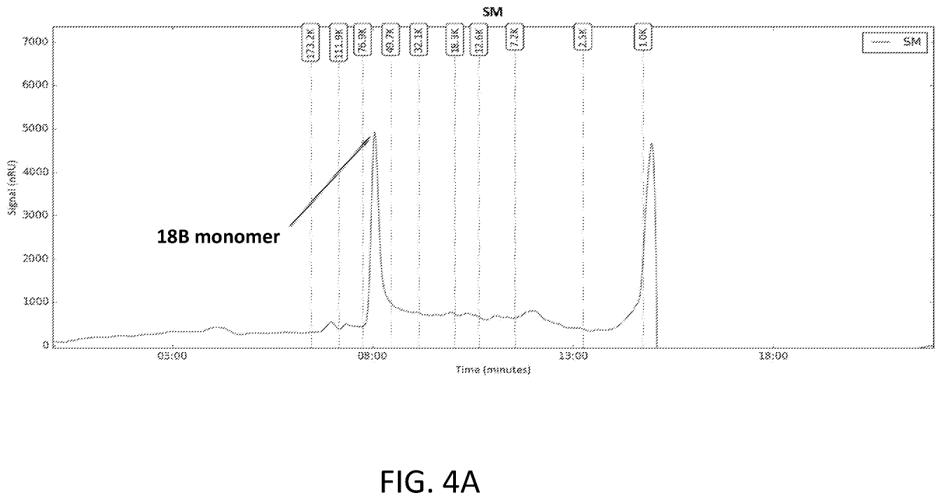

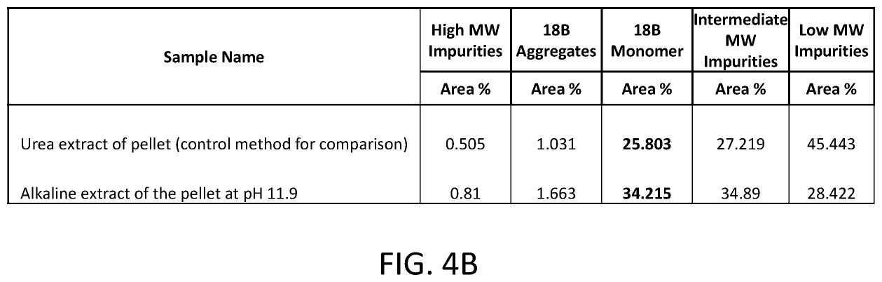

Specifically, cell culture fermentation broth was inoculated with Pichia pastoris expressing 18B recombinant protein and incubated to allow expression of the 18B protein. The cultures were centrifuged to harvest the cells and the cell pellet was re-suspended in distilled water at a ratio of 1:1 (equal amount cell pellet and water) or 1:3 (one part cell pellet and two parts water). The pH of the cell pellet suspension was adjusted with 2-10M NaOH to a final pH of 11.8-11.9. The cell pellet suspension was incubated for 15-30 minutes at room temperature with agitation. The pH was adjusted with NaOH to maintain a pH of 11.8-11.9 during incubation. The cell pellet suspension was centrifuged and the supernatant containing the recombinant protein collected. The supernatant was lyophilized to concentrate the 18B protein and the amount of 18B protein recovered assessed via Size Exclusion Chromatography (SEC) (FIG. 4A and 4B), as described below.

The relative amounts of high, low and intermediate molecular weight impurities, monomeric 18B and aggregate 18B were analyzed using Size Exclusion Chromatography (SEC). 18B powder was dissolved in 5M Guanidine Thiocyanate (GdSCN) and injected onto a Yarra SEC-3000 SEC-HPLC column to separate constituents on the basis of molecular weight. Refractive index was used as the detection modality. 18B aggregates, 18B monomer, low molecular weight (1-8 kDa) impurities, intermediate molecular weight impurities (8-50 kDa) and high molecular weight impurities (110-150 kDa) were quantified. The relevant composition was reported as mass % and area %. BSA was used as a general protein standard with the assumption that >90% of all proteins demonstrate do/dc values (the response factor of refractive index) within ˜7% of each other. Poly(ethylene oxide) was used as a retention time standard, and a BSA calibrator was used as a check standard to ensure consistent performance of the method. As a control, a sample purified using urea to solubilize the 18B protein was also assessed.

The alkaline extraction of 18B from P. pastoris cell pellet at pH 11.9 resulted in 70-75% extraction yield of full length 18B protein normalized to the amount of 18B protein isolated using 5M GdSCN. SEC area % of the extracted 18B protein was used to calculate the purity of the sample. The purity of 18B monomer in the alkaline extracts was about 35% monomer area, 35% intermediate molecular weight impurities area, and 28% low molecular weight impurities % area (FIG. 4A and 4B). In comparison, solubilization of the 18B protein with 10M urea resulted in a lower yield of 18B protein, with about 26% monomer area, 27% intermediate molecular weight impurities area, and 45% low molecular weight impurities area. These data show that the alkaline solubilization and extraction method resulted in greater 18B yield and higher purity of the isolated 18B protein.

### Example 2

### Further Purification of Isolated Silk Polypeptide

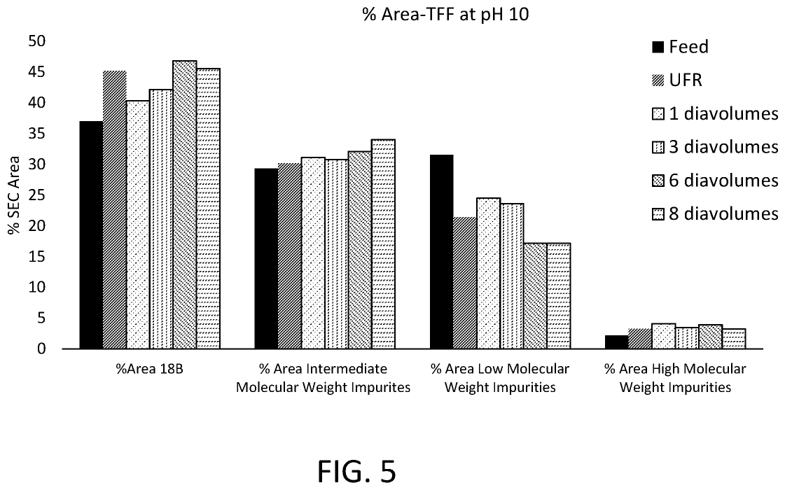

To further purify the 18B spider protein, isolated 18B samples from the alkaline extraction above were subjected to ultrafiltration and tangential flow filtration using a 750 k MW filter and 8 diavolumes of water. The sample comprising the unfiltered protein, the ultrafiltered protein, and the protein after 1, 3, 6, and 8 diavolumes of water was assessed via SEC as previously described. The SEC % area for the 18B monomer, intermediate molecular weight impurities, low molecular weight impurities, and high molecular weight impurities in each sample is shown in FIG. 5. The unfiltered protein sample is shown in the bar on the far left (“Unadjusted feed”), the ultrafiltered protein sample is shown in the bar second to the left (“Unadjusted UFR”), and the 1, 3, 6, or 8 diavolumes samples are shown in the middle left, middle right, second from the right and far right bars, respectively (FIG. 5). Increased diavolumes of washing resulted in increased % area of the 18B monomer and decreased % area of the low molecular weight impurities.

### Example 3

### 18B Purification Using Two-Step Alkaline Extraction

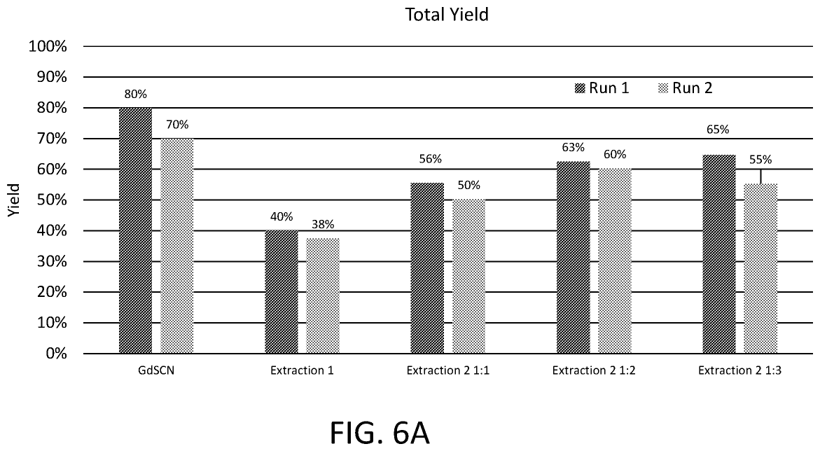

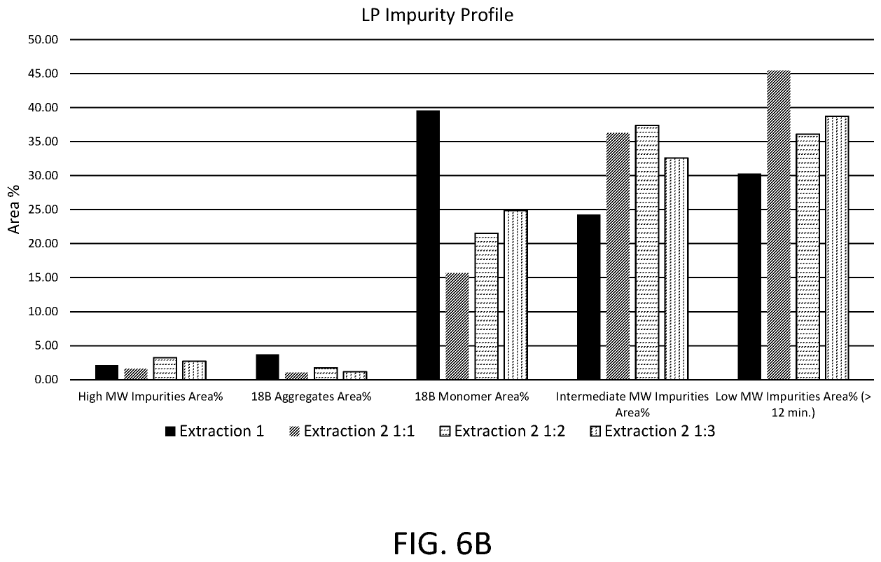

To increase the recovery of the 18B protein from the cells, a two-step extraction process was also performed. The pH of whole cell broth of P. pastoris cells expressing 18B was adjusted to pH 11.8 with 2M NaOH and incubated for 30-60 min as a first alkaline extraction step. A control sample of whole cell broth of P. pastoris cells expressing 18B was incubated with 5M GdSCN for approximately 15 minutes to solubilize and extract the 18B protein. The cells were pelleted, and supernatant collected. The remaining pellets from the first alkaline extraction step were re-extracted by adding water at pH 11.8 in 1:1, 1:2, or 1:3 Pellet:Water ratios as a second extraction step. The supernatant from the first and second alkaline extractions containing the recombinant 18B protein was collected. The supernatant was lyophilized to concentrate the 18B protein and the samples were assessed via SEC as previously described in Example 1. Two separate experimental runs are shown for each extraction condition and the GdSCN control (FIG. 6A). Increasing the amount of alkaline water (1:2 and 1:3 ratios) increased the amount of 18B protein recovered. However, the purity of the double extraction 18B monomer protein was highest on the single extraction. The purity of the 18B monomer also increased as more alkaline water relative to pellet used in the second extraction increased (FIG. 6B).

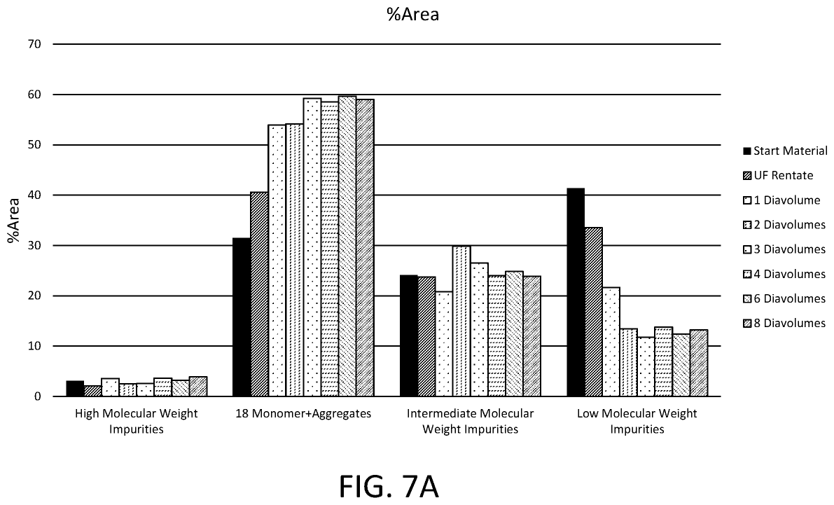

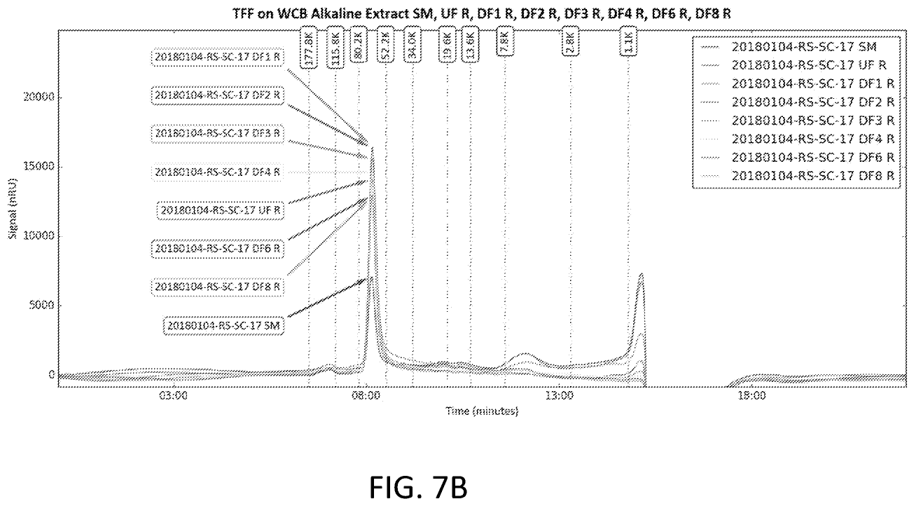

The samples from the extraction were then purified via ultrafiltration and tangential flow filtration using a 750 k MW filter and up to 8 diavolumes of water as previously described. The purity of the resulting silk polypeptide compositions was assessed via SEC (FIG. 7A and 7B). FIG. 7A shows the % area of the 18B monomer, intermediate MW impurities, and low molecular weight impurities. Increased diavolumes during tangential flow filtration resulted in increased 18B monomer peak area. FIG. 7B shows the SEC peaks for each sample, starting material (“SM”), ultrafiltered retentate (“UF R”), and tangential flow filtration diavolume samples 1, 2, 3, 4, 6, and 8 (DF 1, 2,3, 4, 6, 8).

### Example 4

### Further Isolation of Silk Polypeptides from the Alkaline Extract by Altering pH

18B recombinant protein from the alkaline extraction was precipitated from the alkaline extract by adjusting the pH of the extract. In this experiment, alkaline extraction was from a whole cell culture broth was first performed by adjusting the pH of the whole cell culture broth by adding NaOH to a final pH of 11.8-11.9, thereby producing an alkaline cell suspension. The cell suspension was incubated for 15-30 minutes at room temperature with agitation. After incubation, the cell suspension was centrifuged and the alkaline supernatant containing the solubilized 18B protein was collected to generate an 18B alkaline extract.

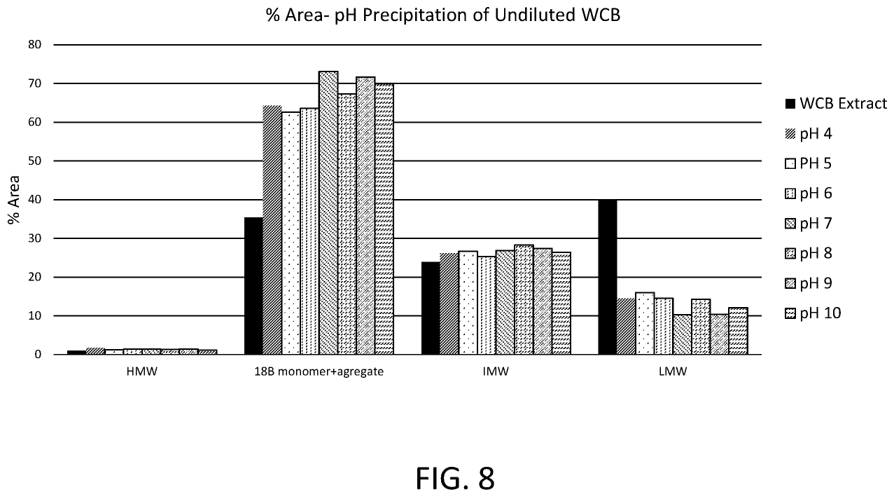

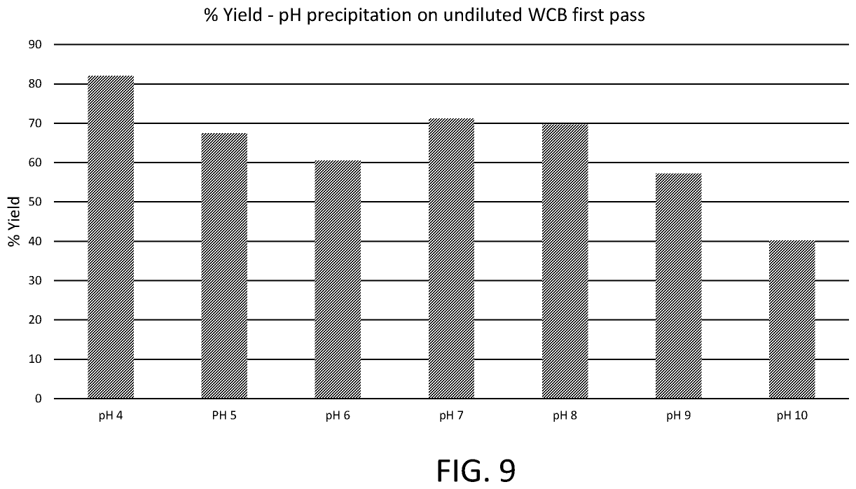

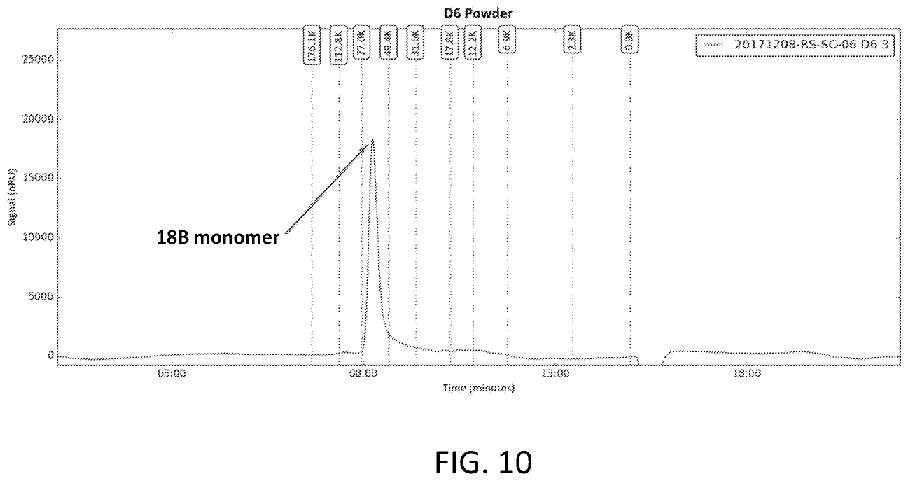

18B alkaline extract samples were then treated with different pH conditions to precipitate the 18B protein. H2504 was added to alkaline extract samples to a final pH of 4, 5, 6, 7, 8, 9, or 10. A precipitate comprising 18B recombinant protein was then isolated from the alkaline extract. The precipitate samples were assessed via SEC as previously described. FIG. 8 shows the SEC % area purity of the high molecular weight (HMW) peak, 18B monomer and aggregate peak, intermediate MW (IMW), and low MW (LMW) peak for each pH condition. FIG. 9 shows the % yield of 18B protein for each precipitant pH tested. Among all the conditions, a pH of 7 for the precipitation step was found to be the most effective for a single stage precipitation method following the initial alkaline extraction of the 18B protein, with ˜70% % area indicating a purity of about 70%. FIG. 10 shows the SEC profile for the 18B precipitate at pH 6.

In addition to diacentrifugation, TFF (tangential flow filtration) was also performed to isolate the alkaline extracts. However, diacentrifugation was more effective than TFF in removing impurities and in general resulted in 60-70% protein recovery with >70% 18B protein purity.

The 18B protein precipitate obtained at pH 6 was lyophilized, wet-spun into a fiber, and subjected to tenacity measurement. The lyophilized 18B protein was dissolved in formic acid to a final protein amount of 36 wt %. The dissolved protein was extruded at 40 μl/min into a 100% ethanol coagulation bath to produce fibers. The 18B fibers produced by this method had a tenacity of 19.4 cN/text.

### Example 5

### P0 Recovery Using Alkaline Conditions vs. Salt Precipitation.

pH buffer solution concentrations and incubation times were tested to determine their use in solubilizing PO (SEQ ID NO: 39) recombinant silk protein in E. coli cell lysate for extraction from a cell culture.

Cell culture fermentation broth was inoculated with E. coli expressing a PO recombinant protein with a C terminal 6x-His tag and incubated to allow expression of the PO protein. The cultures were centrifuged at 15,000 rcf to pellet the cells. The supernatant was removed and the cell pellet was re-suspended in H₂O at a ratio of 1:4 (cell pellet: buffer) or 1:9 (cell pellet: buffer) and incubated for 15-60 minutes. The pH of the re-suspended cell pellet was adjusted with NaOH to a final pH of 9, 10, 10.5 or 11. As a control, a re-suspended cell pellet sample was also incubated with 5M guanidine thiocyanate (GdSCN) and sonicated for 1.5 min. Samples were vortex and homogenized using a rotisserie mixer. The lysate was clarified via centrifugation at 15,000 rcf for 5 minutes and the clarified supernatant containing the PO protein was retained. The supernatant was filtered using a 0.25 μm and analyzed by BCA, ELISA, and immunoblot.

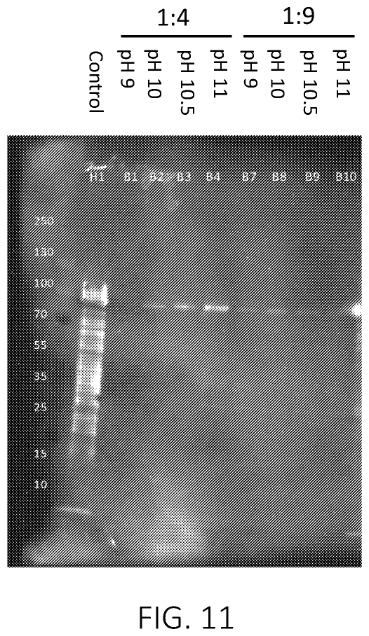

The samples were normalized to 1 mg/ml protein concentration and the amount of solubilized PO in each sample assessed via Western Blot using an anti-His antibody (FIG. 11). Lane H1 is the control sample lysed via sonication in 5M GdSCN. Lanes B1-B4 are the samples mixed at a ratio of 1:4 cell pellet: buffer at at pH 9, pH10, pH 10.5, and pH 11, and lanes B7-B10 are the samples mixed at a ratio of 1:9 cell pellet: buffer at pH 9, pH10, pH 10.5, and pH 11. Lanes C2-C4 are the samples incubated with GdSCN for 15, 30, or 60 minutes.

In an exemplary method, cell culture fermentation broth is inoculated with E. coli expressing PO recombinant protein and incubated to expression of the PO protein. The cultures are centrifuged at 15,000 rcf to pellet the cells. The cell pellet is re-suspended in H₂O at a cell pellet: liquid ratio of 1:1 or 1:3 and the cell suspension is homogenized at 10,000 to 40,000 psi to lyse the E. coli cells. The lysate is clarified via centrifugation and the cell pellet with the insoluble PO is retained. The cell pellet is re-suspended in H₂O and the pH of the cell pellet suspension is adjusted with 2-10M NaOH to a final pH of 11.5. The cell pellet suspension is incubated for 15-60 minutes at room temperature with agitation. The pH is adjusted with NaOH to maintain a pH of 11.5 during incubation. After incubation, the cell suspension is centrifuged and the supernatant containing the recombinant P0 protein was collected.

As an additional method, insoluble PO can also be extracted from the cell pellet using an alkaline buffer with 10M urea. After re-suspension of the cell pellet with H₂O, the pH of the cell pellet suspension is adjusted with 2-10M NaOH to a final pH of 11.5 and urea added to a final concentration of 10M urea. The cell pellet suspension is incubated for 15-60 minutes at room temperature with agitation.

In all methods, the isolated recombinant PO protein can be further purified via additional clarification steps, such as filtration, centrifugation, precipitation, or chromatography.

### Equivalents

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

INFORMAL SEQUENCE LISTING

| SEQ ID | | |
|---|---|---|
| NO | NAME | SEQUENCE |
| 38 | 18B | GGYGPGAGQQGPGSGGQQGPGGQGPYGSGQQGPGGAGQQGPGGQGPYGPGAAAA |
| | | AAAAAGGYGPGAGQQGPGGAGQQGPGSQGPGGQGPYGPGAGQQGPGSQGPGSGG |
| | | QQGPGGQGPYGPSAAAAAAAAAGGYGPGAGQRSQGPGGQGPYGPGAGQQGPGSQ |
| | | GPGSGGQQGPGGQGPYGPSAAAAAAAAGGYGPGAGQQGPGSQGPGSGGQQGPGG |
| | | QGPYGPGAAAAAAAVGGYGPGAGQQGPGSQGPGSGGQQGPGGQGPYGPSAAAAA |
| | | AAAGGYGPGAGQQGPGSQGPGSGGQQGPGGQGPYGPSAAAAAAAAGGYGPGAGQ |
| | | QGPGSGGQQGPGGQGPYGSGQQGPGGAGQQGPGGQGPYGPGAAAAAAAAAGGYG |
| | | PGAGQQPGGAGQQGPGSQGPGGQGPYGPGAGQQGPGSQGPGSGGQQGPGGQGPY |
| | | GPSAAAAAAAAAGGYGPGAGQRSQGPGGQGPYGPGAGQQGPGSQGPGSGGQQGP |
| | | GGQGPYGPSAAAAAAAAGGYGPGAGQQGPGSQGPGSGGQQGPGGQGPYGPGAAA |
| | | AAAAVGGYGPGAGQQGPGSQGPGSGGQQGPGGQGPYGPSAAAAAAAAGGYGPGA |
| | | GQQGPGSQGPGSGGQQGPGGQGPYGPSAAAAAAAAGGYGPGAGQQGPGSGGQQG |
| | | PGGQGPYGSGQQGPGGAGQQGPGGQGPYGPGAAAAAAAAAGGYGPGAGQQGPGG |
| | | AGQQGPGSQGPGGQGPYGPGAGQQGPGSQGPGSGGQQGPGGQGPYGPSAAAAAA |
| | | AAAGGYGPGAGQRSQGPGGQGPYGPGAGQQGPGSQGPGSGGQQGPGGQGPYGPS |
| | | AAAAAAAAGGYGPGAGQQGPGSQGPGSGGQQGPGGQGPYGPGAAAAAAAVGGYG |
| | | PGAGQQGPGSQGPGSGGQQGPGGQGPYGPSAAAAAAAAGGYGPGAGQQGPGSQG |
| | | PGSGGQQGPGGQGPYGPSAAAAAAA |
| 39 | P0 | MAGAGAGYRGQAGYIQGAGASAGAAAAGAGVGYGGQAGYGQGAGASAGAAAAAG |
| | | AGAGRQAGYGQGAGASAGAAAAGAGAGRQAGYGQGAGASAGAAAAGADAGYGGQ |
| | | AGYGQGAGASAGAAASGAGAGYGGQAGYGQGAGASAGAAAAGAGAGYLGQAGYG |
| | | QGAGASAGAAAGAGAGYGGQAGYGQGTGAAASAAASSAGAGAGYRGQAGYIQGA |
| | | GASAGAAAAGAGVGYGGQAGYGQGAGASAGAAAAAGAGAGRQAGYGQGAGASAG |
| | | AAAAGAGAGRQAGYGQGAGASAGAAAAGADAGYGGQAGYGQGAGASAGAAASGA |
| | | GAGYGGQAGYGQGAGASAGAAAAGAGAGYLGQAGYGQGAGASAGAAAGAGAGYG |
| | | GQAGYGQGTGAAASAAASSAGAGAGYRGQAGYIQGAGASAGAAAAGAGVGYGGQ |
| | | AGYGQGAGASAGAAAAAGAGAGRQAGYGQGAGASAGAAAAGAGAGRQAGYGQGA |
| | | GASAGAAAAGADAGYGGQAGYGQGAGASAGAAASGAGAGYGGQAGYGQGAGASA |
| | | GAAAAGAGAGYLGQAGYGQGAGASAGAAAGAGAGYGGQAGYGQGTGAAASAAAS |
| | | SAGAGAGYRGQAGYIQGAGASAGAAAAGAGVGYGGQAGYGQGAGASAGAAAAAG |
| | | AGAGRQAGYGQGAGASAGAAAAGAGAGRQAGYGQGAGASAGAAAAGADAGYGGQ |
| | | AGYGQGAGASAGAAASGAGAGYGGQAGYGQGAGASAGAAAAGAGAGYLGQAGYG |
| | | QGAGASAGAAAGAGAGYGGQAGYGQGTGAAASAAASSAGGGGGG |
| 40 | FLAG | DYKDDDDKDYKDDDDKDYKDDDDK |
| | tag | |

1. A method of isolating recombinant spider silk protein from a host cell culture, comprising:
a. obtaining a cell culture, wherein said cell culture comprises a host cell and a growth medium, wherein said host cell expresses recombinant spider silk protein;
b. collecting a portion of said cell culture comprising said recombinant spider silk protein;
c. incubating said portion of said cell culture in an aqueous solution under alkaline conditions, thereby solubilizing said recombinant spider silk protein in said aqueous solution; and
d. isolating the recombinant spider silk protein from said aqueous solution, thereby producing an isolated recombinant spider silk protein sample.

2. The method of claim 1, wherein the alkaline conditions comprise an alkaline pH from 9 to 14.

3. The method of claim 2, wherein the alkaline pH is from 11 to 12.

4. The method of any of the above claims, wherein the isolated recombinant spider silk protein is a full-length recombinant spider silk protein.

5. The method of claim 4, wherein the isolated recombinant spider silk protein sample comprises at least 30%, at least 35%, at least 40%, at least 45%, at least 50%, at least 55%, at least 60%, at least 65%, at least 70%, at least 75%, at least 80%, at least 85%, at least 90%, or at least 95% full-length recombinant spider silk protein as compared to total isolated recombinant spider silk protein.

6. The method of claim 5, wherein the percentage of full-length recombinant spider silk protein is measured using a Western blot.

7. The method of claim 5, wherein the percentage of full-length recombinant spider silk protein is measured using Size Exclusion Chromatography.

8. The method of any of the above claims, wherein the purity of the isolated recombinant spider silk protein is 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 09-95%, or 95-100%.

9. The method of any of the above claims, wherein the yield of the isolated recombinant spider silk protein is at least 50-55%, 55-60%, 60-65%, 65-70%, 70-75%, 75-80%, 80-85%, 85-90%, 09-95%, or 95-100% as compared to recombinant spider silk isolated by a urea or a guanidine thiocyanate method.

10. The method of any of the above claims, wherein isolating the recombinant spider silk protein comprises precipitating the recombinant spider silk protein by altering said alkaline conditions of said aqueous solution.

11. The method of claim 10, wherein altering said alkaline conditions comprises adjusting the alkaline pH of the portion of the cell culture to a lowered pH from 4 to 10.

12. The method of claim 11, wherein the lowered pH is a pH of 4, 5, 6, 7, 8, 9, or 10.

13. The method of claim 11, wherein the lowered pH is a pH from 6 to 7.

14. The method of any one of claims 10-13, wherein adjusting the alkaline pH comprises adding an acid to the aqueous solution.

15. The method of claim 14, wherein said acid is H₂SO4.

16. The method of any of the above claims, wherein the portion of said cell culture comprises a supernatant, a whole cell broth, or a cell pellet.

17. The method of any of the above claims, wherein collecting said portion of said cell culture comprises removing said host cell from said growth medium and reconstituting said host cell in said aqueous solution.

18. The method of any of the above claims, wherein collecting said portion of said cell culture comprises lysing said host cell.

19. The method of claim 18, wherein lysing comprises heat treatment, shear disruption, physical homogenization, sonication, or chemical homogenization.

20. The method of any of the above claims, wherein said portion of said cell culture comprises said host cell and said growth medium from said cell culture.

21. The method of any of the above claims, wherein said aqueous solution comprises diluted growth medium.

22. The method of any of the above claims wherein incubating said portion of said cell culture under alkaline conditions is performed from 10 to 120 minutes.

23. The method of claim 22, wherein incubating said portion of said cell culture under alkaline conditions is performed for at least 10, at least 15, at least 30, at least 45, at least 60, at least 75, at least 90, at least 105, or at least 120 minutes.

24. The method of claim 22, wherein incubating said portion of said cell culture under alkaline conditions is performed from 15 to 30 minutes.

25. The method of any of the above claims, wherein incubating said portion of said cell culture under alkaline conditions further comprises agitating the portion of the cell culture.

26. The method of any of the above claims, further comprising removing an un-solubilized biomass from said aqueous solution under alkaline conditions.

27. The method of claim 26, wherein removing the un-solubilized biomass comprises filtration, centrifugation, gravitational settling, adsorption, dialysis, or phase separation.

28. The method of claim 27, wherein the filtration is ultrafiltration, microfiltration, or diafiltration.

29. The method of any one of claims 26-28, wherein removing the un-solubilized biomass is repeated at least once.

30. The method of any of the above claims, further comprising removing impurities before isolating the recombinant spider silk protein or after isolating the recombinant spider silk protein.

31. The method of claim 30, wherein removing the impurities comprises filtration, centrifugation, gravitational settling, adsorption, dialysis, or phase separation.

32. The method of claim 31, wherein the filtration is ultrafiltration, microfiltration, or diafiltration.

33. The method of claim 31, wherein the centrifugation is ultracentrifugation or diacentrifugation.

34. The method of claim 31, wherein the adsorption is charcoal adsorption.

35. The method of any one of claims 31-34, wherein removing impurities is repeated at least once.

36. The method of any of the above claims, further comprising concentrating the isolated recombinant spider silk protein to produce a concentrated spider silk protein.

37. The method of claim 36, wherein concentrating comprises precipitation, filtration, ultrafiltration, centrifugation, dialysis, evaporation, or lyophilization.

38. The method of any of the above claims, further comprising drying the isolated recombinant spider silk protein.

39. The method of any of the above claims, further comprising generating a silk fiber from the isolated recombinant spider silk.

40. The method of claim 39, wherein said silk fiber comprises a tenacity of at least 19 cN/tex.

41. The method of any of the above claims, wherein said recombinant spider silk protein is 18B or P0.

42. The method of any of the above claims, wherein the cell culture comprises a fungal, a bacterial or a yeast cell.

43. The method of any of the above claims, wherein the yeast cell is a Pichia pastoris cell.

44. A method of isolating a recombinant spider silk protein, the method comprising
a. obtaining a cell culture, wherein said cell culture comprises a host cell and a growth medium, wherein said host cell expresses a recombinant spider silk protein;
b. collecting a portion of said cell culture comprising said recombinant spider silk protein;
c. incubating said portion of said cell culture in an aqueous solution under alkaline conditions, thereby solubilizing said recombinant spider silk protein in said aqueous solution;
d. adjusting the aqueous solution to a non-alkaline pH, thereby precipitating the said solubilized recombinant spider silk protein; and
e. isolating the recombinant spider silk protein from said portion of cell culture, thereby producing an isolated recombinant spider silk protein.

45. A composition comprising a recombinant spider silk protein produced by the method of any one of the above claims.

46. The composition of claim 45, wherein the recombinant spider silk comprises at least 55%, at least 60%, at least 65%, at least 70%, at least 75%, at least 80%, at least 85%, at least 90%, at least 95%, or 100% full length recombinant spider silk.

47. A silk fiber comprising a recombinant spider silk protein produced by the method of any one of claims 1-44.

48. The silk fiber of claim 47, wherein the silk fiber comprises a tenacity of at least 19 cN/tex.

49. A composition comprising a cell culture comprising a growth medium and a host cell comprising a recombinant spider silk protein in an alkaline buffer solution.

50. The composition of any one of claims 45-49, wherein the alkaline buffer solution has a pH from 9 to 14.

51. The composition of claim 50, wherein the pH is from 11 to 12.

52. The composition of any one of claims 49-51, wherein the spider silk protein is 18B or P0.

53. The composition of claim any one of claims 49-52, wherein the cell culture comprises a fungal, a bacterial, or a yeast cell.

54. The composition of claim 53, wherein the bacterial cell is an E. coli cell.

55. The composition of claim 53, wherein the yeast cell is a Pichia pastoris cell.
